(19) United States
(12) Patent Application Publication
(10) Pub. No.: US 2013/0171719 A1
(43) Pub. Date: Jul. 4, 2013

(54) RECOMBINANT AAV PRODUCTION IN MAMMALIAN CELLS

(71) Applicants: Kyu-Kye Hwang, Gainesville, FL (US); David Knop, Gainesville, FL (US)

(72) Inventors: Kyu-Kye Hwang, Gainesville, FL (US); David Knop, Gainesville, FL (US)

(21) Appl. No.: 13/681,953

(22) Filed: Nov. 20, 2012

Related U.S. Application Data
(63) Continuation of application No. 13/569,744, filed on Aug. 8, 2012, now abandoned.
Application No. 13/569,744 is a continuation of application No. 11/503,775, filed on Aug. 14, 2006, now abandoned.
Application No. 11/503,775 is a continuation-in-part of application No. 10/252,182, filed on Sep. 23, 2002, now Pat. No. 7,091,029.

(51) Int. Cl. C12N 15/864

(52) U.S. Cl. CPC C12N 15/8645
USPC 435/235.1

(57) ABSTRACT

The present invention includes methods and compositions for the production of high titer recombinant Adeno-Associated Virus (rAAV) in a variety of mammalian cells. The disclosed rAAV are useful in gene therapy applications. Disclosed methods based on co-infection of cells with two or more replication-defective recombinant herpes virus (rHSV) vectors are suitable for high-titer, large-scale production of infectious rAAV.

### RELATED APPLICATIONS

The present application is a continuation of U.S. application Ser. No. 13/569,744, filed Aug. 8, 2012, which is a continuation of U.S. application Ser. No. 11/503, 775, filed Aug. 14, 2006, which is a continuation-in-part of U.S. application Ser. No. 10/252,182, entitled High Titer Recombinant AAV Production, filed Sep. 23, 2002, granted. The entire contents of each of the aforementioned applications are hereby incorporated by reference.

### BACKGROUND OF THE INVENTION

The invention is in the field of molecular biology. More specifically, the invention relates to methods for the large-scale production of recombinant adeno-associated virus (rAAV) for use in gene therapy applications.

### DESCRIPTION OF THE RELATED ART

Gene therapy refers to treatment of genetic diseases by replacing, altering, or supplementing a gene responsible for the disease. It is achieved by introduction of a corrective gene or genes into a host cell, generally by means of a vehicle or vector. Gene therapy holds great promise for the treatment of many diseases. Already, some success has been achieved pre-clinically, using recombinant AAV (rAAV) for the delivery and long-term expression of introduced genes into cells in animals, including clinically important non-dividing cells of the brain, liver, skeletal muscle and lung. Clinical trials using this technology have included use of rAAV expressing the cftr gene as a treatment for cystic fibrosis (Flotte et al., 1998; Wagner et al. 1998).

Methods for production of rAAV have been developed in which cells grown in culture are caused to produce rAAV, which is harvested from the cells and purified. Production methods for rAAV typically require the presence of three necessary elements in the cells: 1) a gene of interest flanked by AAV inverted terminal repeat (ITR) sequences, 2) AAV rep and cap genes, and 3) helper virus proteins (“helper functions”). Conventional protocols for production of rAAV include delivering the first two elements by transfection of the cells with plasmid DNA containing the appropriate recombinant gene cassettes. The helper functions have traditionally been supplied by infecting the cells with a helper virus such as adenovirus (Ad) (Samulski et al., 1998; Hauswirth et al., 2000).

Despite the potential benefits of gene therapy as a treatment for human diseases, unfortunately, a serious practical limitation stands in the way of its widespread use in the clinic. It has been estimated that in order to produce even a single clinically effective dose for a human patient, over 10¹⁴ rAAV particles must be made (Snyder, et al., 1997; Ye et al., 1999). On a commercial scale, the required level of cell culture poses a serious practical barrier to large-scale production of rAAV in “cell factories,” or bioreactors. Thus, it is recognized that the benefits of improving rAAV infectious particle yield per cell will be very significant from a commercial production standpoint. For example, an improvement resulting in a two-fold increase in rAAV yield per cell would allow for culture of half as many cells. A ten-fold increase would enable the same amount of rAAV product to be made by one-tenth the number of producer cells. Significant improvements of this magnitude are desirable in order to achieve economic feasibility for this technology.

Conventional AAV production methodologies make use of procedures known to limit the number of rAAV that a single producer cell can make. The first of these is transfection using plasmids for delivery of DNA to the cells. It is well known that plasmid transfection is an inherently inefficient process requiring high genome copies and therefore large amounts of DNA (Hauswirth et al., 2000). Additionally, use of Ad significantly reduces the final rAAV titers because it is a contaminant that must be removed from the final product. Not only must effective procedures be employed to eliminate Ad contamination, but stringent assays for Ad contamination of rAAV are also necessary. Purification and safety procedures dictated by the use of Ad result in loss of rAAV at each step.

Advances toward achieving the desired goal of scalable production systems that can yield large quantities of clinical grade rAAV vectors have largely been made in production systems that utilize transfection as a means of delivering the genetic elements needed for rAAV production in a cell. For example, losses during down-stream purification associated with removal of contaminating adenovirus have been circumvented by replacing adenovirus infection with plasmid transfection in a three-plasmid transfection system in which a third plasmid comprises nucleic acid sequences encoding adenovirus helper proteins (Xiao et al. 1998). Improvements in two-plasmid transfection systems have also simplified the production process and increased rAAV vector production efficiency (Grimm et al., 1998). Despite these advances, it is generally recognized that transfection systems are limited in their efficiency by the uptake of exogenous DNA, and in their commercial utility due to scaling difficulties.

Several strategies for improving yields of rAAV from cultured mammalian cells are based on the development of specialized producer cells created by genetic engineering. In one approach, production of rAAV on a large scale has been accomplished by using genetically engineered “proviral” cell lines in which an inserted AAV genome can be “rescued” by infecting the cell with adenovirus or HSV. Proviral cell lines can be rescued by simple adenovirus infection, offering increased efficiency relative to transfection protocols. However, as with the earlier transfection methods, adenovirus is introduced into the system that must later be removed. Additionally, the rAAV yield is generally low in proviral cell lines (Qiao et al. 2002a).

There are several further disadvantages that limit approaches using proviral cell lines. The cell cloning and selection process itself can be laborious; additionally, this process must be carried out to generate a unique cell line for each therapeutic gene of interest (GOI). Furthermore, cell clones having inserts of unpredictable stability can be generated from proviral cell lines.

A second cell-based approach to improving yields of rAAV from cells involves the use of genetically engineered “packaging” cell lines that harbor in their genomes either the AAV rep and cap genes, or both the rep-cap and the ITR-gene of interest (Qiao et al., 2002b). In the former approach, in order to produce rAAV, a packaging cell line is either infected or transfected with helper functions, and with the AAV ITR-GOI elements. The latter approach entails infection or transfection of the cells with only the helper functions. Typically, rAAV production using a packaging cell line is initiated by infecting the cells with wild-type adenovirus, or recombinant adenovirus. Because the packaging cells comprise the rep and cap genes, it is not necessary to supply these elements exogenously.

While rAAV yields from packaging cell lines have been shown to be higher than those obtained by proviral cell line rescue or transfection protocols, packaging cell lines typically suffer from recombination events, such as recombination of E1a-deleted adenovirus vector with host 293 cell DNA. Infection with recombinant adenovirus therefore initiates both rAAV production and generation of replication-competent adenovirus. Furthermore, only limited success has been achieved in creating packaging cell lines with stable genetic inserts.

Recent progress in improving yields of rAAV has also been made using approaches based on delivery of helper functions from herpes simplex virus (HSV) using recombinant HSV amplicon systems. Although modest levels of rAAV vector yield, of the order of 150-500 viral genomes (v.g.) per cell, were initially reported (Conway et al., 1997), more recent improvements in rHSV amplicon-based systems have provided substantially higher yields of rAAV v.g. and infectious particles (i.p.) per cell (Feudner et al., 2002).

Amplicon systems are inherently replication-deficient; however the use of a “gutted” vector, replication-competent (rcHSV), or replication-deficient rHSV still introduces immunogenic HSV components into rAAV production systems. Therefore, appropriate assays for these components and corresponding purification protocols for their removal must be implemented. Additionally, amplicon stocks are difficult to generate in high titer, and often contain substantial parental virus contamination.

It is apparent from the foregoing that there is a clear need for improved large-scale methods for production of high titer, infectious rAAV to overcome the major barrier to the routine use of rAAV for gene therapy.

### SUMMARY OF THE INVENTION

The present invention seeks to overcome some of the deficiencies in the prior art by addressing problems that limit production of rAAV in sufficient quantities for efficient gene therapy procedures. Using methods and materials disclosed herein, high titers of infectious rAAV can be obtained in a variety of mammalian cell lines including those that have not been genetically altered by recombinant genetic engineering for improved rAAV production. In some instances, the yields of infectious rAAV particles per cell are at least an order of magnitude greater than previously reported for the same cell types using other rAAV production strategies.

The invention is based on a novel method for producing high titer rAAV as described in co-pending U.S. Patent Application No. 0/252,182. In the method, mammalian cells are simultaneously or sequentially within several hours co-infected with at least two recombinant herpes simplex viruses (rHSV). The two rHSV are vectors designed to provide the cells, upon infection, with all of the components necessary to produce rAAV. The method does not require the use of mammalian cells specialized for expression of particular gene products. This is advantageous because the invention can be practiced using any mammalian cell generally suitable for this purpose. Examples of suitable genetically unmodified mammalian cells include but are not limited to cell lines such as HEK-293 (293), Vero, RD, BHK-21, HT-1080, A549, Cos-7, ARPE-19, and MRC-5.

Accordingly, and in one aspect, the invention provides a method for producing high titer recombinant Adeno-Associated Virus (rAAV) in a mammalian cell, comprising: (a) infecting a mammalian cell with (i) a first replication-defective recombinant herpes simplex virus (rHSV) comprising a nucleic acid including an AAV rep gene and an AAV cap gene operably linked to a promoter; and (ii) a second replication-defective recombinant herpes simplex virus (rHSV) comprising a nucleic acid including inverted terminal repeat sequences (ITRs) and a gene of interest, such as a gene encoding a therapeutically useful protein, operably linked to a promoter. The mammalian cell is incubated following infection with the rHSV, and rAAV is obtained from the cell. The titer of rAAV produced by the cell using the inventive method varies depending upon the type of cell used for rAAV production, with yields ranging from about 1000 to over 9000 infectious particles (i.p.) per cell.

In one embodiment of the method the mammalian cell is a 293 cell and extremely high titers (up to 9000 i.p. per cell or more) can be obtained. In these preparations, the ratio of vector genomes to infectious particles (v.g.:i.p.) is about 15:1 Other embodiments yielding high titer rAAV on a large scale are based on BHK and Cos-7 cells, in which titers of about 6500-6700 i.p. per cell are obtainable. Lower yields, in the range of 2100 i.p. per cell can be obtained in Vero cells, and in the range of 1600 i.p. per cell for HT 1080 cells, which may be desirable for commercial rAAV production due to characteristics other than titer alone, such as lack of tumorigenicity.

Many embodiments of the rAAV production method utilize mammalian cells that are genetically unmodified, including 293, Vero, RD, BHK, HT 1080, A549, Cos-7, ARPE-19 and MRC-5 cells.

Any rHSV suitable for the purpose can be used in the invention. Embodiments of the rHSV used in the invention can be replication-defective. Infection of producer cells with rHSV that is incapable of replication is preferred because in contrast to methods involving use of adenovirus (Ad), the rHSV does not become a significant contaminant of the rAAV product. This increases the final yield of rAAV by eliminating purification steps associated with removal of Ad.

In a particular embodiment of the invention, a replication-defective rHSV is based on a mutant of HSV-1 comprising a mutation in the ICP27 gene. Any other suitable mutants of HSV exhibiting a replication-defective phenotype can also be used to construct the rHSV.

In one embodiment, a first replication-defective rHSV comprises a nucleic acid including an AAV rep gene and an AAV cap gene, operably linked to a promoter. Other rHSV vectors can be used, such as rHSV comprising a nucleic acid encoding either rep or cap sequences.

Embodiments of the first rHSV of the method include but are not limited to gene constructs based on variants of the cap gene found in various serotypes of AAV, including AAV-1, AAV-2, AAV-3, AAV-4, AAV-5 and AAV-6, AAV-7 and AAV-8. Also within the scope of the invention are novel AAV serotypes, and those modified by recombination or mutation of existing serotypes.

In certain embodiments, nucleic acids encoding AAV rep and cap sequences in the first rHSV are operably linked to their native promoters. In other embodiments, heterologous promoters are used to direct expression of the AAV nucleic acid sequences. Non-limiting examples of other promoters that can be used in the disclosed method include but are not limited to an SV40 early promoter, a CMV promoter, a Herpes tk promoter, a metallothionine inducible promoter, a mouse mammary tumor virus promoter and a chicken β-actin promoter.

In one preferred embodiment, the rep-cap encoding nucleic acid construct in the first rHSV is inserted into the tk gene of rHSV virus. Any other suitable site or sites in the HSV genome may be used for integration of the rep and cap encoding nucleic acid sequences.

The second replication-defective rHSV of the invention comprises inverted terminal repeats (ITRs) from AAV and one or more genes of interest (GOI), the expression of which is directed by one or more promoters. In some embodiments, the gene of interest is inserted between a pair of ITRs. The GOI may be a gene likely to be of therapeutic value. Examples of therapeutic genes include but are not limited to α-1 antitrypsin, GAA, erythropoietin and PEDF.

When it is desirable to select for or to identify successful transgene expression, the GOI may be a reporter gene. Many examples of genes used as reporters or for selection are known, and can be used in the invention. These include but are not limited to the genes encoding beta-galactosidase, neomycin, phosphoro-transferase, chloramphenicol acetyl transferase, thymidine kinase, luciferase, beta-glucuronidase, aminoglycoside, phosphotransferase, hygromycin B, xanthine-guanine phosphoribosyl, luciferase, DHFR/methotrexate, and green fluorescent protein (GFP).

In another aspect, the invention provides a method for producing high-titer rAAV in a mammalian cell. The titer of rAAV, as determined by v.p:i.p. per cell, is at least 3-fold higher than the titer obtained in the same mammalian cell by a rAAV production method that does not involve co-infection with rHSV.

The timing of co-infection with the first and second rHSV in the rHSV-based, Ad-free system for rAAV production is an important factor that can affect the yield of infectious rAAV per cell. Highest yields of infectious rAAV are obtained in cells that are simultaneously infected, or serially infected with two different rHSV within several hours. Serial infection at longer intervals is at best about 35% as effective as simultaneous co-infection, and at worst results in negligible production of rAAV. Other factors affecting yields include the relative proportions of the first and second rHSV, the duration of incubation times following simultaneous co-infection, choice of producer cells, and culture conditions employed both for producer cells and cells used for titration of rAAV stocks.

The invention is the first to utilize co-infection of producer cells with at least two different replication-defective rHSV vectors to achieve production of rAAV. An unexpectedly high yield of rAAV is achieved through the use of simultaneous infection of producer cells with the rHSVs, as opposed to adding the two rHSVs at different times. The effect of timing of rHSV co-infection on rAAV yields is an important discovery of the invention. It is shown that deviation from the simultaneous co-infection protocol is markedly detrimental to the rAAV yield. For example, introduction of a delay of 4 hours between infections with the first and second rHSV results in a reduction to about 35% of the level of rAAV produced by the simultaneous co-infection protocol. With delays of 12 and 24 hours between infections, production of rAAV drops to insignificant levels.

Another factor in maximizing rAAV production is the ratio of the two rHSV viruses used in the simultaneous co-infection procedure. In a particular embodiment of the invention in which the first rHSV was rHSV/rc and the second rHSV was rHSV/AAV-GFP, best results were obtained when the ratio of the first rHSV to the second rHSV was about 6:1. This ratio is likely to differ with other rHSV used in the invention, and may be determined experimentally with each combination of first and second rHSV selected for use.

Methods of the invention described herein utilize simultaneous co-infection with at least two rHSVs to deliver the minimal set of components required for rAAV production in mammalian cells. Those of skill in the art will recognize that the disclosed simultaneous co-infection method can be modified to include further steps designed to deliver other components to the cells. Examples of such further steps include, but are not limited to, e.g., infection with at least one other virus, including 1) other rHSV differing in construction from the first and second rHSV, or 2) other strains of naturally occurring or recombinant viruses such as Ad, rAAV, Ad, or recombinant Ad (rAd). Infection with the additional virus can be either simultaneous with the co-infection with the first and second rHSV, or may be carried out either before or after the simultaneous co-infection with the first and second rHSV. Alternatively, or in addition to, the step of infection with at least one additional virus, the method can include an additional step involving transfection with at least one plasmid DNA, including an AAV expression vector, so long as a simultaneous co-infection step is performed.

It is contemplated that the gain in efficiency of rAAV yield per cell achievable using the disclosed methods and compositions of the invention will be particularly advantageous for the commercial production of rAAV. By providing in some cases the benefit of at least ten-fold reduction in the requirements for cell culture, the invention offers the potential for significant savings in facilities producing rAAV on the scale needed for therapeutic use in gene therapy.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIGS. 1A-D are four schematic drawings illustrating genetic components of recombinant herpes simplex virus (rHSV) vectors useful for production of recombinant adeno-associated virus (rAAV), in accordance with an embodiment of the invention.

FIG. 2 is a graph showing comparative rAAV production data using simultaneous co-infection and single infection protocols, in accordance with an embodiment of the invention.

FIG. 3 is a graph showing the effect on rAAV production of varying the timing of addition of rHSV/rc and rHSV/GFP viruses to the cells.

FIGS. 4A-B are graphs showing the effect on rAAV production of varying the proportion of rHSV/rc (R) (FIG. 4A) and rHSV/GFP (G) (FIG. 4B) in the co-infection protocol.

FIG. 5 is a graph showing the effect on rAAV production of varying the timing of harvest of the producer cells.

FIG. 6 is a graph showing the effect of seeding density of producer cells (293) on production of rAAV.

FIG. 7 is a graph showing the effect of seeding density of C12 cells on quantification of rAAV/GFP.

FIG. 8 is a graph showing production of rAAV as a function of MOI ratio of the first and second rHSV, in accordance with an embodiment of the invention.

### DETAILED DESCRIPTION

### Definitions

As used herein, the term “infection” refers to delivery of heterologous DNA into a cell by a virus. The term “simultaneous co-infection” denotes simultaneous infection of a producer cell with at least two viruses. The meaning of the term “co-infection” as used herein means “double infection,” “multiple infection,” or “serial infection” but is not used to denote simultaneous infection with two or more viruses. Infection of a producer cell with two (or more) viruses at different times will be referred to as “co-infection.” The term “transfection” refers to a process of delivering heterologous DNA to a cell by physical or chemical methods, such as plasmid DNA, which is transferred into the cell by means of electroporation, calcium phosphate precipitation, or other methods well known in the art.

As used herein, the term “transgene” refers to a heterologous gene, or recombinant construct of multiple genes (“gene cassette”) in a vector, which is transduced into a cell. Use of the term “transgene” encompasses both introduction of the gene or gene cassette for purposes of correcting a gene defect in the cell for purposes of gene therapy, and introduction of the gene or gene cassette into a producer cell for purposes of enabling the cell to produce rAAV. By the term “vector” is meant a recombinant plasmid or viral construct used as a vehicle for introduction of transgenes into cells.

The terms “recombinant HSV,” “rHSV,” and “rHSV vector” refer to isolated, genetically modified forms of herpes simplex virus (HSV) containing heterologous genes incorporated into the viral genome. By the term “rHSV/rc” or “rHSV/rc virus” is meant a rHSV in which the AAV rep and cap genes have been incorporated into the rHSV genome. The terms “rHSV expression virus,” and “rHSV/AAV” denote a rHSV in which inverted terminal repeat (ITR) sequences from AAV have been incorporated into the rHSV genome. The terms “rHSV/AAV-GFP” and “rHSV/GFP” refer to an rHSV/AAV in which the DNA sequence encoding green fluorescent protein (GFP) has been incorporated into the viral genome.

The term “producer cell” refers any cell line, either genetically unmodified, or genetically modified, that is used for production of rAAV. Heterologous genes needed for rAAV production by the producer cell are typically introduced by viral infection, or by transfection, e.g., with plasmid DNA. Preferred cell lines useful for production of rAAV by infection with rHSV as described herein include, but are not limited to, 293, 293-GFP and Vero cells. The 293-GFP cell line is a genetically modified 293-derived cell line, produced from plasmid pTR-UFS, in which the AAV-2 ITRs and GFP, driven by a CMV promotor, have been integrated into the genome of the cells (Conway et al., 1997).

The term “AAV-GFP” refers to an infectious recombinant AAV particle containing a heterologous gene, i.e., GFP.

The term “gene of interest” (GOI) is meant to refer to a heterologous sequence introduced into an AAV expression vector, and typically refers to a nucleic acid sequence encoding a protein of therapeutic use in humans or animals, or a reporter protein useful for detecting expression of the GOI by the rAAV, inserted between AAV inverted terminal repeat sequences.

Gene Therapy Using rAAV Vectors.

The invention provides a novel method of producing recombinant adeno-associated virus (rAAV). Recent efforts to use rAAV as a vehicle for gene therapy hold promise for its applicability as a treatment for human diseases based on genetic defects. The ability of rAAV vectors to integrate into the chromosomes of host cells makes it possible for rAAV to mediate long-term, high level expression of the introduced genes. An additional advantage of rAAV is its ability to perform this function in non-dividing cell types including hepatocytes, neurons and skeletal myocytes. rAAV has been used successfully as a gene therapy vehicle to enable expression of erythropoietin in skeletal muscle of mice (Kessler et al., 1996), tyrosine hydroxylase and aromatic amino acid decarboxylase in the CNS in monkey models of Parkinson disease (Kaplitt et al., 1994) and Factor IX in skeletal muscle and liver in animal models of hemophilia. At the clinical level, the rAAV vector has been used in human clinical trials to deliver the cftr gene to cystic fibrosis patients and the Factor IX gene to hemophilia patients (Flotte, et al., 1998, Wagner et al, 1998).

Required Elements of rAAV Production Systems.

Recombinant AAV is produced in vitro by introduction of gene constructs into cells known as producer cells. Known systems for production of rAAV employ three fundamental elements: 1) a gene cassette containing the gene of interest, 2) a gene cassette containing AAV rep and cap genes and 3) a source of “helper” virus proteins.

The first gene cassette is constructed with the gene of interest flanked by inverted terminal repeats (ITRs) from AAV. ITRs function to direct integration of the gene of interest into the host cell genome. (Hermonat and Muzyczka, 1984, Samulski, et al., 1983). The second gene cassette contains rep and cap, AAV genes encoding proteins needed for replication and packaging of rAAV. The rep gene encodes four proteins (Rep 78, 68, 52 and 40) required for DNA replication. The cap genes encode three structural proteins (VP1, VP2, and VP3) that make up the virus capsid (Muzyczka and Berns, 2001.)

The third element is required because AAV-2 does not replicate on its own. Helper functions are protein products from helper DNA viruses that create a cellular environment conducive to efficient replication and packaging of rAAV. Adenovirus (Ad) has been used almost exclusively to provide helper functions for rAAV. The gene products provided by Ad are encoded by the genes E1a, E1b, E2a, E4orf6, and Va (Samulski et al., 1998; Hauswirth et al., 2000; Muzyczka and Burns, 2001.)

Production Technologies for rAAV.

Production of rAAV vectors for gene therapy is carried out in vitro, using suitable producer cell lines such as 293 and HeLa. A well known strategy for delivering all of the required elements for rAAV production utilizes two plasmids and a helper virus. This method relies on transfection of the producer cells with plasmids containing gene cassettes encoding the necessary gene products, as well as infection of the cells with Ad to provide the helper functions. This system employs plasmids with two different gene cassettes. The first is a proviral plasmid encoding the recombinant DNA to be packaged as rAAV. The second is a plasmid encoding the rep and cap genes. To introduce these various elements into the cells, the cells are infected with Ad as well as transfected with the two plasmids. Alternatively, in more recent protocols, the Ad infection step can be replaced by transfection with an adenovirus “helper plasmid” containing the VA, E2A and E4 genes (Xiao, et al., 1998, Matsushita, et al., 1998).

While Ad has been used conventionally as the helper virus for rAAV production, it is known that other DNA viruses, such as Herpes simplex virus type 1 (HSV-1) can be used as well. The minimal set of HSV-1 genes required for AAV-2 replication and packaging has been identified, and includes the early genes UL5, UL8, UL52 and UL29 (Muzyczka and Burns, 2001). These genes encode components of the HSV-1 core replication machinery, i.e., the helicase, primase, primase accessory proteins, and the single-stranded DNA binding protein (Knipe, 1989; Weller, 1991). This rAAV helper property of HSV-1 has been utilized in the design and construction of a recombinant Herpes virus vector capable of providing helper virus gene products needed for rAAV production (Conway et al., 1999).

Quantitative Limitations of Current rAAV Production Techniques.

Efficient, large scale production of rAAV, as discussed above, will be necessary in order for gene therapy to become a practical treatment for human disease. It is estimated that for clinical effectiveness, over 10¹⁴ particles per dose of rAAV will be necessary for most applications (Snyder, et al., 1997, Ye et al., 1999). Conventional rAAV techniques involving plasmid transfection are capable of producing approximately 500 rAAV particles per cell (Conway et al., 1997).

The most advanced production systems for rAAV, including Ad-free transfection based methods, rep and cap inducible cell lines, and the use of recombinant adenovirus or recombinant Herpes virus are reported to produce approximately 5×10⁴ particles of rAAV per cell (Conway et al., 1999, Xiao, et al., 1998, Matsushita, et al., 1998, Gao et al., 1998). To determine the number of infective particles per cell, this number must be reduced by about one hundred fold. The actual number of infectious particles per cell is typically about two orders of magnitude lower than the total number of particles per cell, assuming a typical particle to infectivity ratio of 100:1. Therefore, even the most advanced production techniques typically produce about 500 infectious particles per cell. Using any of the rAAV production protocols currently known, at least 2×10⁹ cells would have to be infected or transfected to produce 10¹⁴ particles of rAAV. Thus to produce sufficient infectious rAAV for even one dose using current methodology, it would be necessary to culture over 2×10¹¹ cells (approximately 6500 tissue culture flasks). This level of cell culture surpasses what realistically can be accomplished using standard laboratory tissue culture methods, and is the most serious practical barrier to large-scale commercial production of rAAV.

Recombinant Herpes Virus-Based Simultaneous Co-Infection Protocol for rAAV Production: An Overview.

The invention provides a novel Ad-free, transfection-free method of making rAAV, based on the use of two or more recombinant rHSV viruses used to co-infect producer cells with all of the components necessary for rAAV production. It is possible to use HSV-1, an alternate DNA helper virus of AAV, in lieu of Ad to provide the helper functions needed for rAAV production. Like Ad, HSV-1 is able to fully support AAV replication and packaging (Knipe, 1989, Knipe, 1989, Buller, 1981, Mishra and Rose, 1990, Weindler et al., 1991, Johnson et al., 1997). The minimal set of HSV-1 genes required to replicate and package AAV is UL5, UL8, UL52 and UL29 (Weindler et al., 1991). These genes encode components of the HSV-1 core replication machinery and by themselves form nuclear prereplication centers that develop into mature replication foci (Weindler et al., 1991, Knipe, D. M. 1989). In the present invention, recombinant HSV-1 viruses are used to supply the helper functions needed for rAAV production.

Amplicon systems typically require co-infection of cells with a replication-deficient rHSV vector that provides helper functions for rAAV production. The invention provides a simplified rHSV-based system for rAAV production that uses two or more replication-deficient rHSV vectors including one for the delivery of the rAAV rep and cap functionalities and one for delivery of the gene of interest (GOI) flanked by the inverted terminal repeats (ITR-GOI). Advantageously, the availability of separate replication-defective rHSV vectors of the invention as described makes it possible to modulate the rep and cap functionalities relative to the GOI, by varying the co-infection multiplicity of infection (MOI).

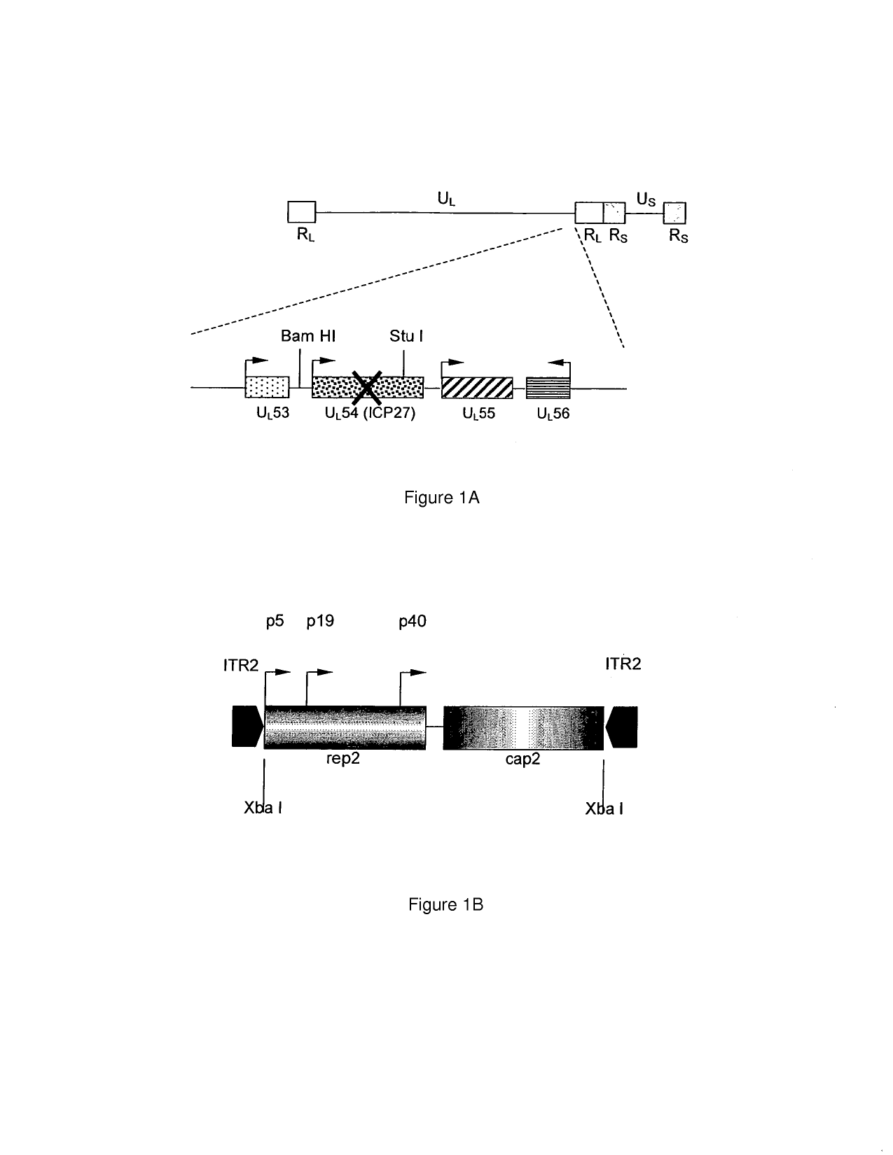

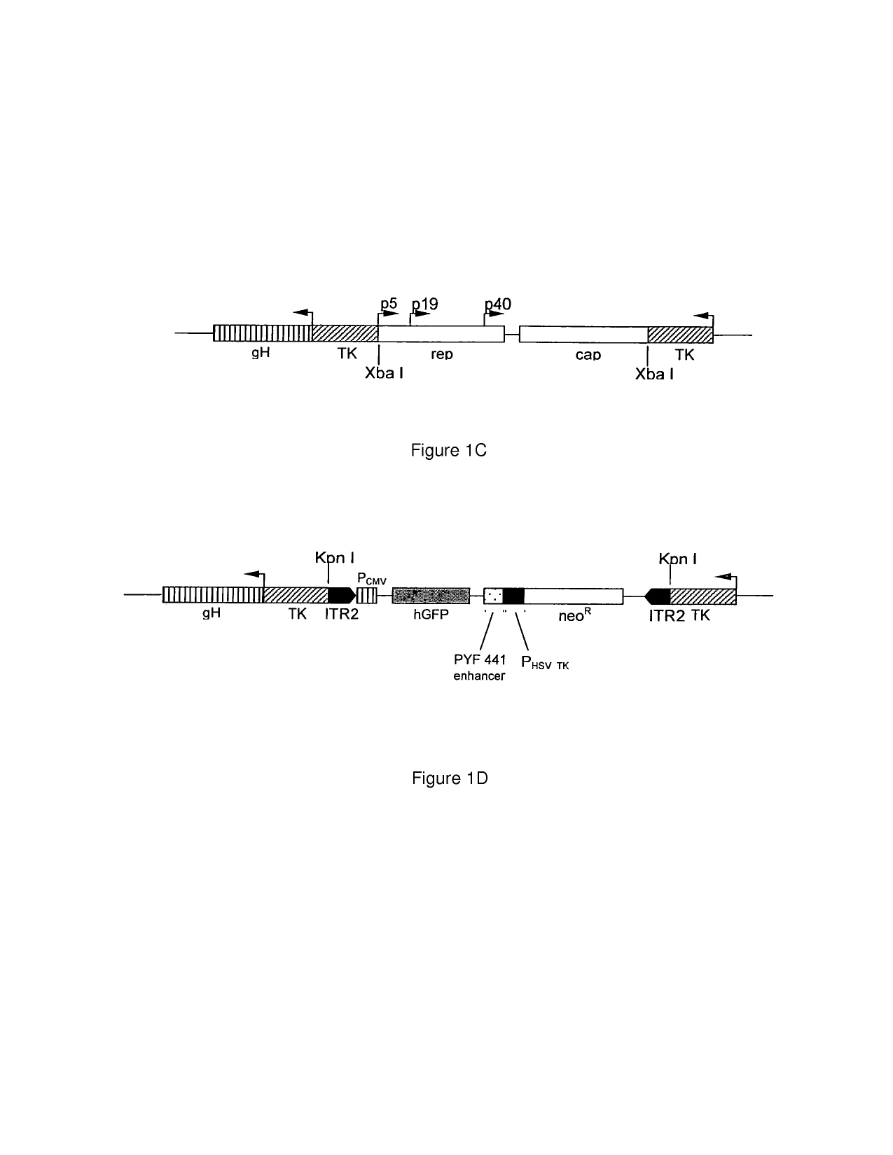

Exemplary genetic sequences for rHSV vectors and rAAV vectors useful for understanding the co-infection method are shown diagrammatically in FIGS. 1A-D. Referring to FIG. 1A, the “X” indicates the site of the ICP27 (U_{L}54) deficiency located between Bam HI and Stu I restriction sites in the rHSV vector backbones. FIG. 1B illustrates the wild-type AAV-2 genome. FIG. 1C illustrates the location of a rep2/cap2 cassette within the thymidine kinase (TK) gene of an exemplary embodiment of a rHSV vector comprising AAV-2 rep and cap sequences. FIG. 1D illustrates an exemplary second rHSV vector comprising a cassette that includes a gene of interest (in this case, humanized green fluorescent protein, hGFP). As shown in the diagram, in this embodiment the AAV2 ITR-GFP gene cassette is also inserted into the TK gene.

The disclosed methods employ simultaneous use of at least two different forms of rHSV, each containing a different gene cassette, as discussed. In addition to supplying the necessary helper functions, each of these rHSV viruses is engineered to deliver different AAV (and other) genes to the producer cells upon infection. The two rHSV forms used in the invention are referred to as the “rHSV/rc virus” and the “rHSV expression virus.” The two are designed to perform different, yet complementary functions resulting in production of rAAV.

The rHSV/rc virus contains a gene cassette in which the rep and cap genes from AAV are inserted into the HSV genome. The rep genes are responsible for replication and packaging of the rAAV genome in host cells infected with AAV. The cap genes encode proteins that comprise the capsid of the rAAV produced by the infected cells. The rHSV/rc virus is used therefore to enable the producer cells to make the protein products of the AAV rep and cap genes.

The second recombinant HSV used in the invention is an “rHSV expression virus.” A usual element of an rAAV production system is an expression cassette (or “expression vector”) containing transgene DNA sequences encoding a gene(s) of interest, along with promoter elements necessary for expression of the gene. Expression vectors engineered for rAAV production are generally constructed with the GOI inserted between two AAV-2 inverted terminal repeats (ITRs). The ITRs are responsible for the ability of native AAV to insert its DNA into the genome of host cells upon infection, or otherwise persist in the infected cells.

In conventional methods, the expression cassette (containing the AAV ITRs, GOI, and a promoter) is delivered to the producer cells by way of transfection with plasmid DNA that includes such constructs. Alternatively, the expression cassette is integrated into the genome of a specialized producer cell line, such as, e.g., the 293-GFP. In the latter case, only helper functions need to be added to the producer cells in order to rescue the foreign DNA from the host cell genome, making it available for packaging into rAAV particles containing the recombinant DNA.

In contrast to these approaches, in the methods of the present invention, the expression cassette is incorporated into a second rHSV-1 virus, i.e., the rHSV expression virus described above. This second rHSV virus is used for co-infection of the cells along with the rHSV-1/rc virus. In a particular embodiment of the rHSV expression virus useful as a marker of gene expression and described in the examples below, the expression cassette contains green fluorescent protein (GFP) as the gene of interest, driven by a CMV promotor. This embodiment of the rHSV expression virus is herein referred to as “rHSV/AAV/GFP,” or simply “rHSV/GFP.”One advantage of a strategy of using two or more rHSV viruses is that both the need for transfection and the need for a specialized producer cell line are eliminated.

High Titer Production of rAAV Using rHSV-Based Co-Infection Protocols.

The invention provides a novel rHSV-based method for production of high titer rAAV. Following co-infection of producer cells with two rHSV viruses, all of the components required for production of infectious rAAV particles are delivered to the cells without the need for transfection, a step known to reduce efficiency of rAAV production. Additionally, use of rHSV for provision of helper functions obviates the requirement for Ad, a helper virus conventionally used for this purpose. Thus two significant problems associated with previous rAAV production protocols are eliminated by the disclosed method.

Production levels of rAAV of up to at least 6000-7000 i.p./cell were achieved using this method. In the development of the present invention, the production of rAAV was investigated using a simultaneous co-infection protocol of the invention. In some assays, the experimental design involved a comparison of the level of rAAV produced by two methods—1) the simultaneous co-infection method and 2) a method involving single infection with rHSV/rc. In a typical assay of this type, replicate cultures of unmodified producer cells (e.g. 293) were simultaneously co-infected with rHSV/rc and an rHSV expression virus (rHSV/GFP), whereas replicate cultures of 293-GFP (having AAV-GFP integrated into the cellular genome) were singly infected with only rHSV/rc.

Under identical experimental conditions, results consistently demonstrated that the simultaneous co-infection method was at least twice as effective as the single infection method. The numbers of infectious rAAV produced per cell by the simultaneous co-infection protocol ranged from about 2300-6000 i.p./cell. In contrast, under the same conditions, the range following single infection was from about 1200-1600 i.p./cell.

These production figures exceed those commonly obtained using even the most advanced production methods (Clark, 2002). For example, previous use of d27.1-rc, which is comparable to the rHSV/rc of the invention, resulted in 380 expression units (EU) of AAV-GFP produced from 293 cells following transfection with AAV-GFP plasmid DNA, and up to 480 EU/cell when the producer cell was GFP-92, a proviral 293-derived cell line (Conway et al., 1999). By contrast, results obtained using the method of the invention were an order of magnitude greater than this.

Studies described herein revealed that a number of experimental variables affected the production of rAAV using the co-infection method. Of particular note was the observation that simultaneous co-infection with the two viruses, i.e., rHSV/rc and the rHSV expression virus was far superior to double or multiple infection with the same viruses (i.e., infection with the first rHSV, followed by infection with the second rHSV after an interval of hours, e.g., 4-24). These experiments revealed the importance of the timing of the addition of the two viruses, demonstrating the clear superiority of co-infection over double infection, even with delays of as little as four hours between addition of the first and the second rHSV.

The relative amounts of the first and second viruses added at the time of simultaneous co-infection also had a pronounced effect on rAAV production. Best results were obtained when the ratio of a first virus (rHSV/rc) to a second virus (rHSV/GFP) was about 6:1.

Another parameter that significantly affects yields of rAAV in the co-infection protocol is the choice of cell line used for production of rAAV. Experiments designed to test two cell lines commonly used for rAAV production, i.e., 293 and Vero cells, demonstrated that of the two, 293 was clearly the cell line of choice, producing about 5 times the amount of rAAV as Vero cells grown, infected and harvested under the same conditions. Other cell lines shown herein to produce high titer rAAV include, e.g., BHK and Cos-7.

Other variables that significantly affect yields of rAAV include the initial plating density of the producer cell line (e.g., 293) and the time of harvest of the producer cells.

Construction of Recombinant HSV-1 Viruses.

The invention utilizes two or more rHSV viruses in a co-infection protocol to produce rAAV. Methods of making rHSV from HSV-1 are generally known in the art (Conway et al., 1999).

rHSV/rc. In one embodiment of the invention, a recombinant HSV designated rHSV/rc was used to demonstrate the efficacy of the novel rAAV production method. This virus was based on a recombinant vector expressing the AAV-2 rep and cap genes in a mutant HSV-1 vector designated d27.1 (Rice and Knipe, 1990) and was prepared as previously described (Conway et al., 1999). As a result of the mutation, this vector does not produce ICP27. An advantage in the use of an ICP27 mutant for rAAV production is that host cell splicing of messenger RNA is known to be inhibited by ICP27 (Sandri-Goldin and Mendoza, 1992). ICP27 probably also effects the appropriate splicing of the AAV-2 rep and cap messages. This vector was chosen because it is replication-defective and was expected to show reduced cytotoxicity compared with wild type (wt) HSV-1. in a non-permissive cell line.

The virus d27.1 displays several other features that make its use advantageous for the design of a helper virus for rAAV production. First, it expresses the early genes known to be required for rAAV production (Weindler et al., 1991, Rice and Knipe, 1990). In addition, d27.1 over-expresses ICP8, the single-stranded DNA binding protein that is the product of UL29, one of the HSV-1 genes essential for AAV replication and packaging (Weindler et al., 1991, Rice and Knipe, 1990, McCarthy, et al., 1989). Increased expression of ICP8 would therefore be predicted to augment rAAV production.

In one embodiment of the HSV/rc vector used in the invention, the AAV-2 rep and cap genes are expressed under control of their native promoters. The p5 and p19 promoters of AAV-2 control expression of Rep 78 and 68, and Rep 52 and 40, respectively. The p40 promoter controls expression of VP1, VP2 and VP3. It will be apparent to those of skill in the art that any other promotor suitable for the purpose can be used and is also within the scope of the invention. Examples of other suitable promoters include SV40 early promoter, and Herpes tk promoter, metallothianine inducible promoter, mouse mammary tumor virus promoter and chicken β-actin promoter.

rHSV expression virus. The rHSV-1 expression virus of the invention was produced in much the same manner as rHSV/rc, by homologous recombination into the HSV-1 tk gene, starting, e.g., with plasmids pHSV-106 and plasmid pTR-UFS. The latter is an AAV proviral construct with AAV-2 ITRs flanking both a humanized GFP and a neomycin resistance gene (neo) expression cassette, in which expression of the GFP is driven by the human CMV promotor (Conway et al., 1999). rHSV/GFP contains a CMV driven gfp expression cassette inside the AAV ITRs and was recombined into the tk locus of the virus d27.1-lacz.

Recombinant HSV Viruses Based on AAV Capsids from AAV-1, AAV-3 or AAV-4 Serotypes.

The invention includes a method for producing rAAV particles with capsid proteins expressed in multiple serotypes of AAV. This is achieved by co-infection of producer cells with a rHSV expression virus and with a rHSV/rc helper virus in which the cap gene products are derived from serotypes of AAV other than, or in addition to, AAV-2. Recombinant AAV vectors have generally been based on AAV-2 capsids. It has recently been demonstrated that rAAV vectors based on capsids from AAV-1, AAV-3, or AAV-4 serotypes differ substantially from AAV-2 in their tropism.

Capsids from other AAV serotypes offer advantages in certain in vivo applications over rAAV vectors based on the AAV-2 capsid. First, the appropriate use of rAAV vectors with particular serotypes may increase the efficiency of gene delivery in vivo to certain target cells that are poorly infected, or not infected at all, by AAV-2 based vectors. Secondly, it may be advantageous to use rAAV vectors based on other AAV serotypes if re-administration of rAAV vector becomes clinically necessary. It has been demonstrated that re-administration of the same rAAV vector with the same capsid can be ineffective, possibly due to the generation of neutralizing antibodies generated to the vector (Xiao, et al., 1999, Halbert, et al., 1997). This problem may be avoided by administration of a rAAV particle whose capsid is composed of proteins from a different AAV serotype, not affected by the presence of a neutralizing antibody to the first rAAV vector (Xiao, et al., 1999). For the above reasons, recombinant AAV vectors constructed using cap genes from serotypes other than, or in addition to, AAV-2 are desirable.

It will be recognized that the construction of recombinant HSV vectors similar to rHSV/rc but encoding the cap genes from other AAV serotypes (e.g. AAV-1, AAV-3 to AAV-8) is achievable using the methods described herein to produce rHSV/rc. The significant advantages of construction of these additional rHSV vectors are ease and savings of time, compared with alternative methods used for the large-scale production of rAAV. In particular, the difficult process of constructing new rep and cap inducible cell lines for each different capsid serotypes is avoided.

Highly Productive rHSV-Based rAAV Manufacturing Process.

The invention provides a rAAV production method based on co-infection with two or more rHSV that features the advantages of flexibility, scalability, and high yield of infectious rAAV. The rHSV vectors used are readily propagated to high titer on permissive cell lines both in tissue culture flasks and bioreactors. The exemplary ICP27-deficient rHSV vectors afford a unique rAAV production environment, permitting high-titer rAAV production of, e.g., about 6400 ip/cell with a low vg:ip of 15. The co-infection method results in substantially higher i.p./cell yields and lower v.g.:i.p. ratios than other known production protocols.

### EXAMPLES

The invention is further described in the following examples, which do not limit the scope of the invention described in the claims.

### Example 1

### Materials and Methods

Recombinant HSV viruses. A recombinant HSV-1 helper virus, designated rHSV/rc, containing AAV-2 rep and cap genes, was constructed by homologous recombination techniques as previously described for a rHSV-1 vector designated d27.1-rc, (Conway et al., 1999). A second rHSV-1, which is a rHSV expression virus designated rHSV/GFP, containing AAV-2 ITRs flanking humanized GFP, was constructed as follows.

Cell Lines For rAAV Production And Titering. Vero, 293 and C12 cell lines were obtained from American Type Culture Collection (Rockville, Md.). Cell lines used for production of rAAV by infection with rHSV, defined herein as “producer cells,” include inter alia 293, 293-GFP and Vero cells.

Choice Of Producer Cells For rHSV Single And Co-infection Protocols. In examples described herein involving production of rAAV by producer cells, the co-infection technique using two rHSV to deliver all of the components required for rAAV production was compared with a single infection technique using only rHSV/rc. In the single infection protocol, the infections were carried out using the 293-GFP cell line, in which the protein of interest (GFP) is already present within the genetic makeup of the cells, as described above. Thus, the producer cells for the single infection protocol were 293-GFP, whereas for the double infection protocols, the producer cells were unmodified 293 cells, complemented by supplying the GFP expression cassette in the second rHSV, i.e., rHSV/GFP. For both single and double infection protocols, the cell lines (either 293 or 293-GFP or Vero) were plated at the same density (generally 1×10⁷ cells per T75 flask) and otherwise treated the same. In experiments designed to test the effect of varying 293 plating density, cells were seeded at initial plating densities of 0.5, 0.7, 1.0, 1.5 and 2.0×10⁷ cells/flask.

Infection of Producer Cells With rHSV and Recovery of rAAV. Viruses used in the infection procedures were diluted from stock preparations to desired concentrations in DMEM, then added to the flasks containing 293, 293-GFP, or Vero producer cells. At the time of addition of the viruses, which was generally on the next day after plating, the cells were approximately 70-80% confluent. Titers of stock preparations of rHSV/rc and rHSV/GFP were in the range of 5×10⁷-1×10⁸ infectious particles (i.p.)/ml. In some of the double infection protocols, varying proportions of rHSV/rc to rHSV/GFP were added, with the multiplicity of infection (MOI) of the two recombinant viruses ranging as follows: rc/GFP: 8/0.7, 8/1, 8/1.5, 8/2, 8/3, 4/1.5, 6/1.5, 12/1.5, and 16/1.5. In other experiments using the double infection protocol, optimal timing of addition of the two viruses was tested. In these experiments, rHSV/rc and rHSV/GFP were added to the 293 cells at different intervals rather than simultaneously. In a typical experiment, the two viruses were added to the cells either simultaneously, or with a delay of 4, 8 or 24 hours between the addition of the first and second virus. The effect of delaying the addition of either virus was tested, i.e., with either rHSV/rc or rHSV/GFP being added first.

Following an incubation interval, the virus-infected cells were harvested and pelleted. The cell pellet was then resuspended in 10 ml of DMEM and cell-associated rAAV was recovered from the producer cells by lysis of the cells using standard techniques involving three rounds of freezing and thawing (Conway et al., 1999). The cell lysates were then titrated for quantification of infectious units of AAV-GFP. In experiments designed to test the optimal time of harvest, producer cells were harvested at various intervals (22, 26, 30, 34, 46 hours) after infection.

Assay of Infectious rAAV. The C12 cell line is a HeLa-derived cell line with inducible AAV-2 rep gene expression (Clark et al., 1995). This cell line was employed in experiments used to assay the number of infectious rAAV particles produced by the production methods of the invention. For this purpose, C12 cells were generally seeded in 96-well plates at densities of 1.2-1.6×10⁴ cells/well. In some experiments designed to test the effect of C12 seeding density, a range of higher plating densities (2.4, 3.3, 4.2×10⁴ cell/well) was used. The amount of AAV-GFP produced was determined using a fluorescent cell assay by titering the virus in the cell lysate by serial dilutions on C12 cells in 96 well plates after co-infection with adenovirus (MOI of 20) and counting fluorescent cells by fluorescence microscopy. The fluorescent assay used for this purpose has been previously described (Conway et al., 1999; Zolotukhin et al., 1999). The viral yield per cell was then calculated and the most efficient MOI was determined.

### Example 2

### Comparison of rAAV Production Levels Using Simultaneous Co-Infection and Single Infection

This example describes a novel adenovirus-free, transfection-free method of producing infectious rAAV particles using simultaneous co-infection of 293 cells with two recombinant HSV-1 viruses, rHSV/rc and rHSV/GFP, and demonstrates the superiority of the new method over a single infection protocol using rHSV/rc alone in producer cells having an integrated AAV-GFP expression cassette inserted into the genome.

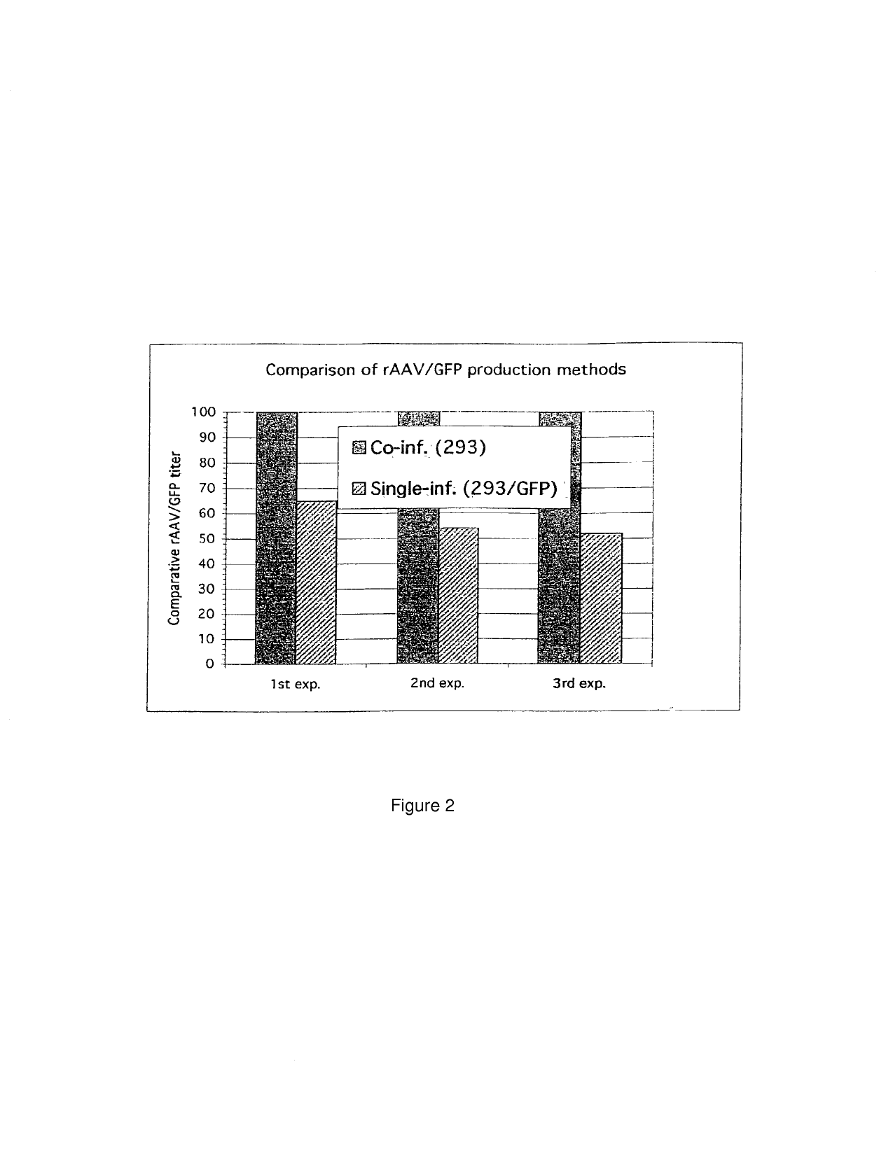

Assays were performed in which production of rAAV was compared using the single infection and co-infection protocols described in Example 1 above. FIG. 2 shows results from three separate experiments in which 293 or 293-GFP cells were plated concurrently at the same seeding density, and either singly infected with rHSV/rc (293-GFP cells) or co-infected with rHSV/rc and rHSV/GFP (293 cells). Following harvest and preparation of cell lysates containing rAAV-GFP produced by the two methods, C12 cells were infected with the rAAV-GFP and the numbers of infectious rAAV-GFP were determined. Results showed that under the identical conditions of the experiment, the simultaneous co-infection protocol was much more effective than single infection with only rHSV/rc. rAAV yields in the three experiments were 2300, 2600, and 2420 i.p./cell using the co-infection protocol, vs. 1600, 1400 and 1260, respectively for the single infection method. With the level of production using co-infection normalized to 100%, production using single infection was found to range from a low of about 52% to a high of about 65% of that obtained by co-infection (FIG. 2).

### Example 3

### Co-Infection: Effect of Timing of Virus Infection

The above example demonstrates the superiority of a simultaneous co-infection protocol using two recombinant rHSV (rHSV/rc and rHSV/GFP) over single infection using only rHSV to deliver the rep and cap genes to the producer cells. This example, involving a co-infection protocol using rHSV/rc and rHSV/GFP, shows the effect of varying the time of infection with each of the recombinant viruses.

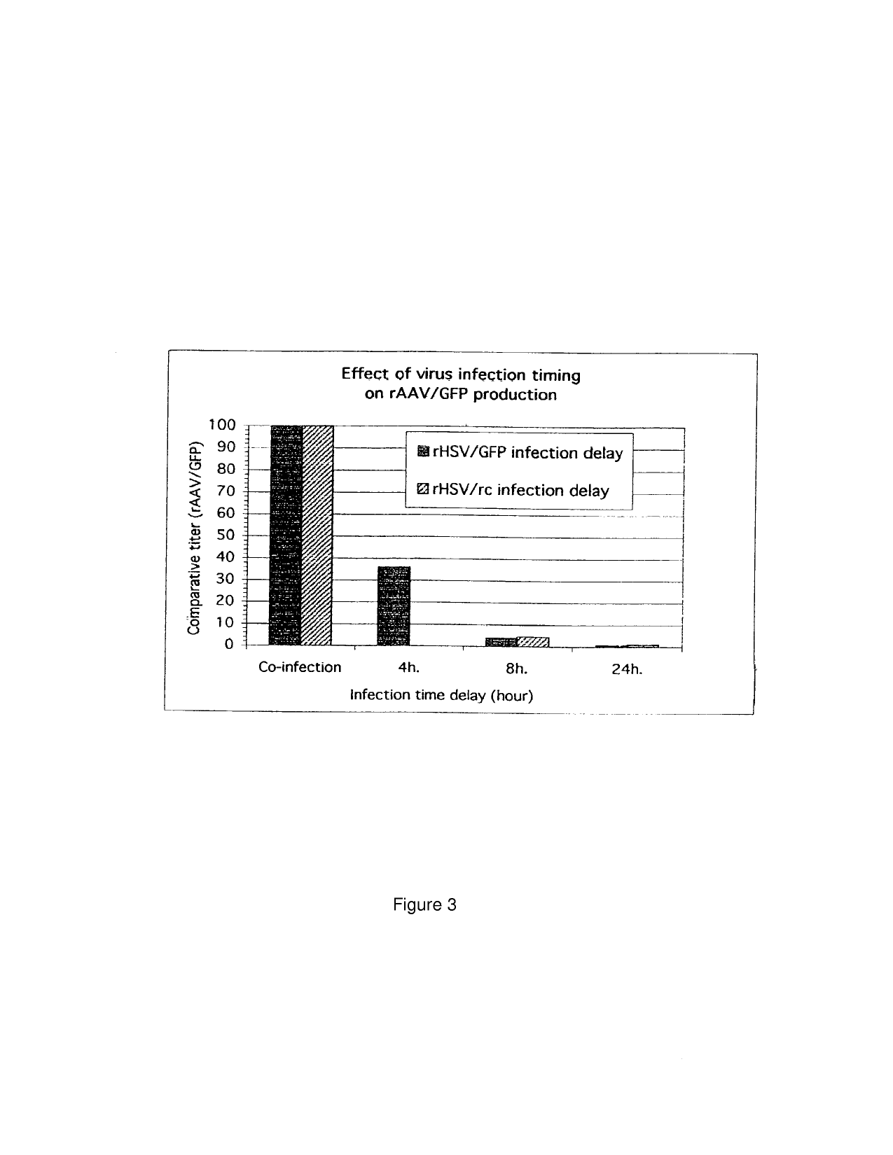

The experiments were carried out by either co-infection of replicate cultures of 293 cells with rHSV/rc and rHSV/GFP, or by double infection of the cells with one of the two viruses (at time 0) and addition of the other after an interval of 4, 8 or 24 hours. FIG. 3 shows results demonstrating that co-infection was markedly superior to multiple infection at each of the times indicated. With addition of rHSV/rc first, followed by rHSV/GFP after a delay of 4 hours, yield of rAAV dropped to about 30% of the value obtained by co-infection (590 vs. 1940 i.p./cell). With longer delays of 8 hours and 24 hours, production of rAAV was negligible (74 and 14 i.p./cell, respectively). Similar results were obtained when rHSV/GFP was added first, and rHSV/rc was added after a delay of 8 or 24 hours. In that case as well, production of rAAV was insignificant compared with the simultaneous co-infection values (86 and 20 i.p./cell, vs. 1940 i.p./cell) (FIG. 3).

### Example 4

### Simultaneous Co-Infection: Effect of Varying rHSV Ratios

The previous example shows that co-infection is superior to multiple infection using two recombinant HSV viruses for production of rAAV in producer cells. This example, using simultaneous co-infection with rHSV/rc and rHSV/GFP, demonstrates the effect of varying the relative proportions of the two viruses in the co-infection procedure. All procedures were as described. For simplicity, the ratio of rHSV/rc to rHSV/GFP is abbreviated to “R/G.”

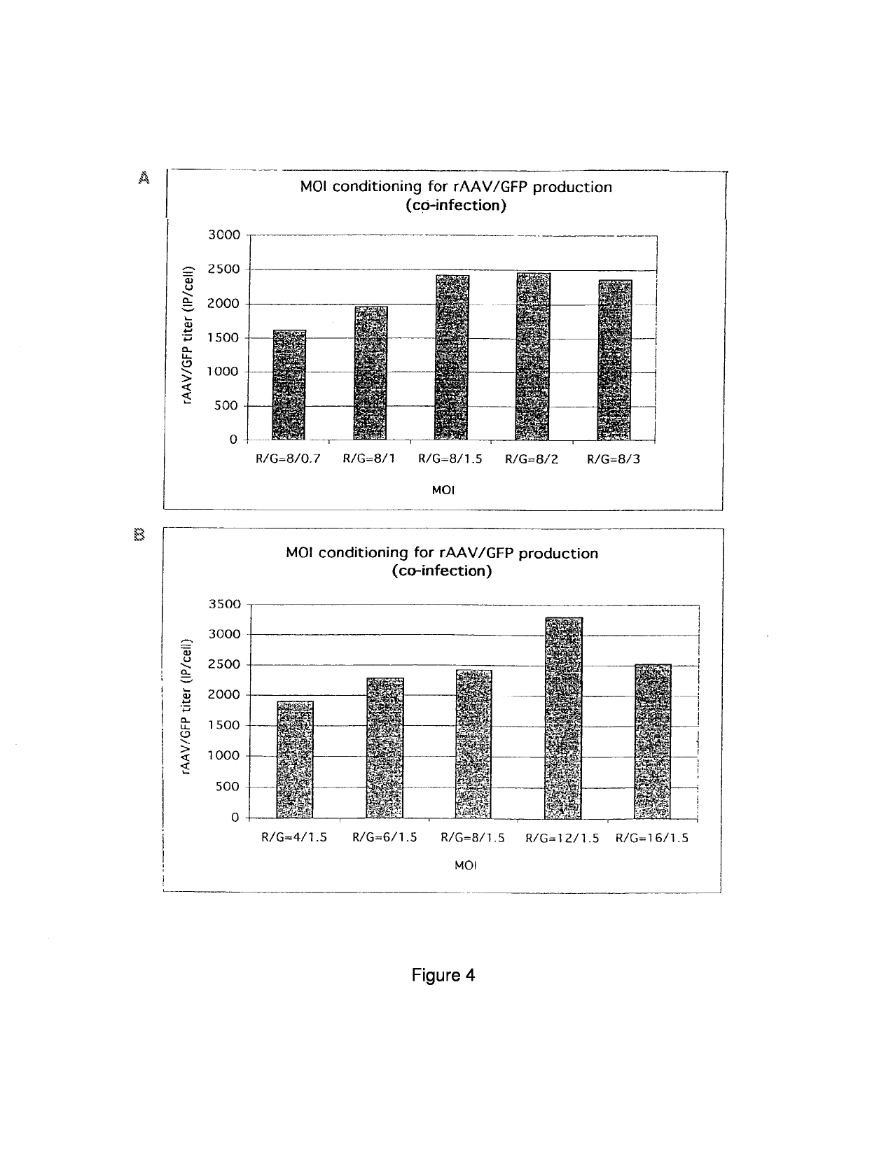

FIGS. 4A and B show data from two experiments in which the R/G ratio was varied, in all cases with the value for R being higher than that for G. The values for the R/G ratio varied from a low of (8/0.7) to a high of (8/2). Results from this assay showed that best production occurred when the R/G ratio was 8:1, with a MOI of 12 and 1.5, respectively for R and G.

### Example 5

### Simultaneous Co-Infection: Effect of Time of Harvest

This example demonstrates that the choice of timing for harvest of the producer cells can affect the yield of rAAV.

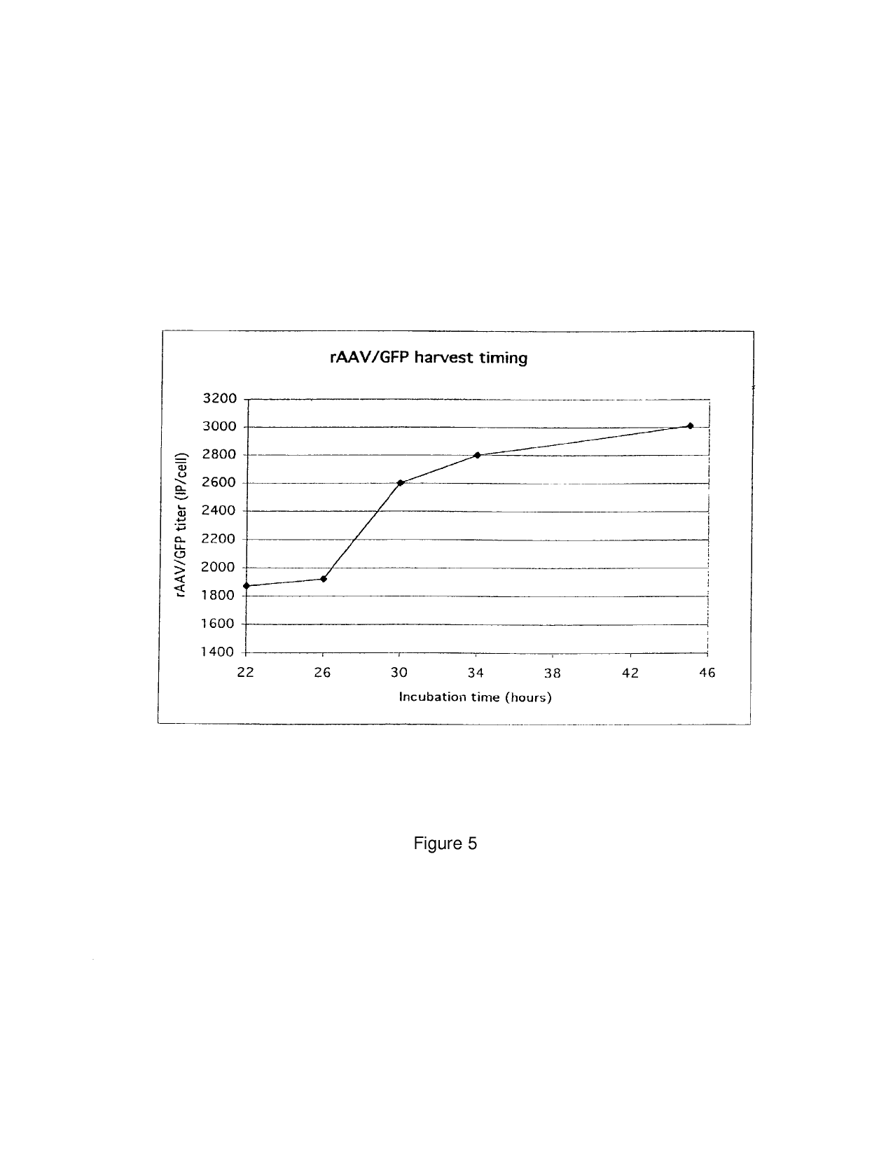

Assays were carried out as described, on replicate cultures of 293 cells co-infected under the same conditions with identical concentrations of R/G. Only the time of harvest was varied, from 22 to 46 hours after co-infection. Results of this assay (FIG. 5) revealed that highest yields of rAAV are obtained when the incubation period before harvest was 46 hours. When cell harvesting was performed between 22 and 26 hours after co-infection, the yield of rAAV-GFP was approximately 1900 infectious particles (i.p.) per cell. In contrast, delay of harvest to 26, 34 and 46 hours after co-infection resulted in improvements in yield of about 2600, 2800 and 3000 i.p./cell, respectively (FIG. 5).

### Example 6

### Simultaneous Co-Infection: Effect of 293 Cell Seeding Density

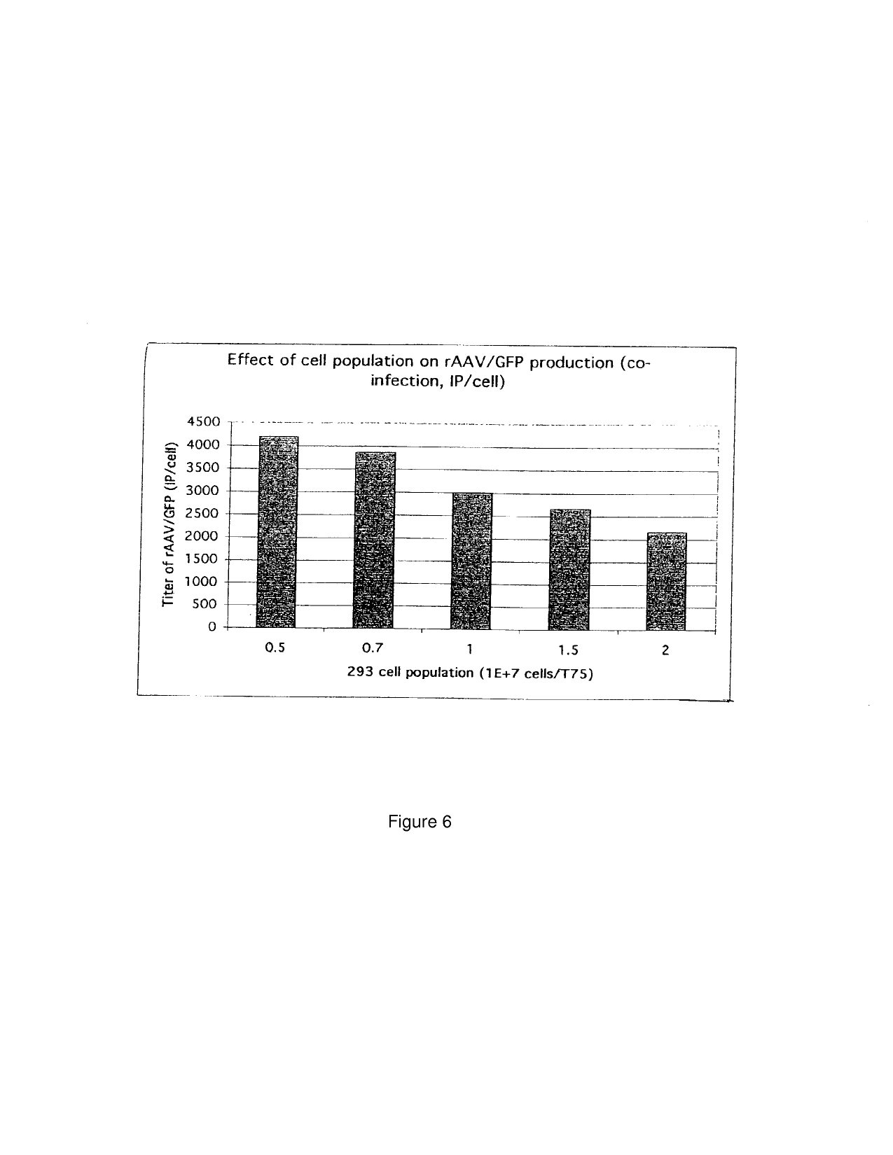

To determine the effect of seeding density of the producer cells on rAAV-GFP production, 293 cells were plated at five seeding densities ranging from 0.5-2.0×10⁷ cells per T75 flask. Following co-infection with rHSV/rc and rHSV/GFP, cells were harvested and rAAV production was quantitated. Results showed a progressive decline in production of rAAV at each of the seeding densities above 0.5×10⁷ cells per flask (FIG. 6). In the experiments shown, production values for 0.5, 0.7, 1.0, 1.5 and 2.0×10⁷ were 4200, 3860, 3000, 2660, and 2160 i.p./cell.

### Example 7

### Simultaneous Co-Infection: Effect of C12 Cell Density

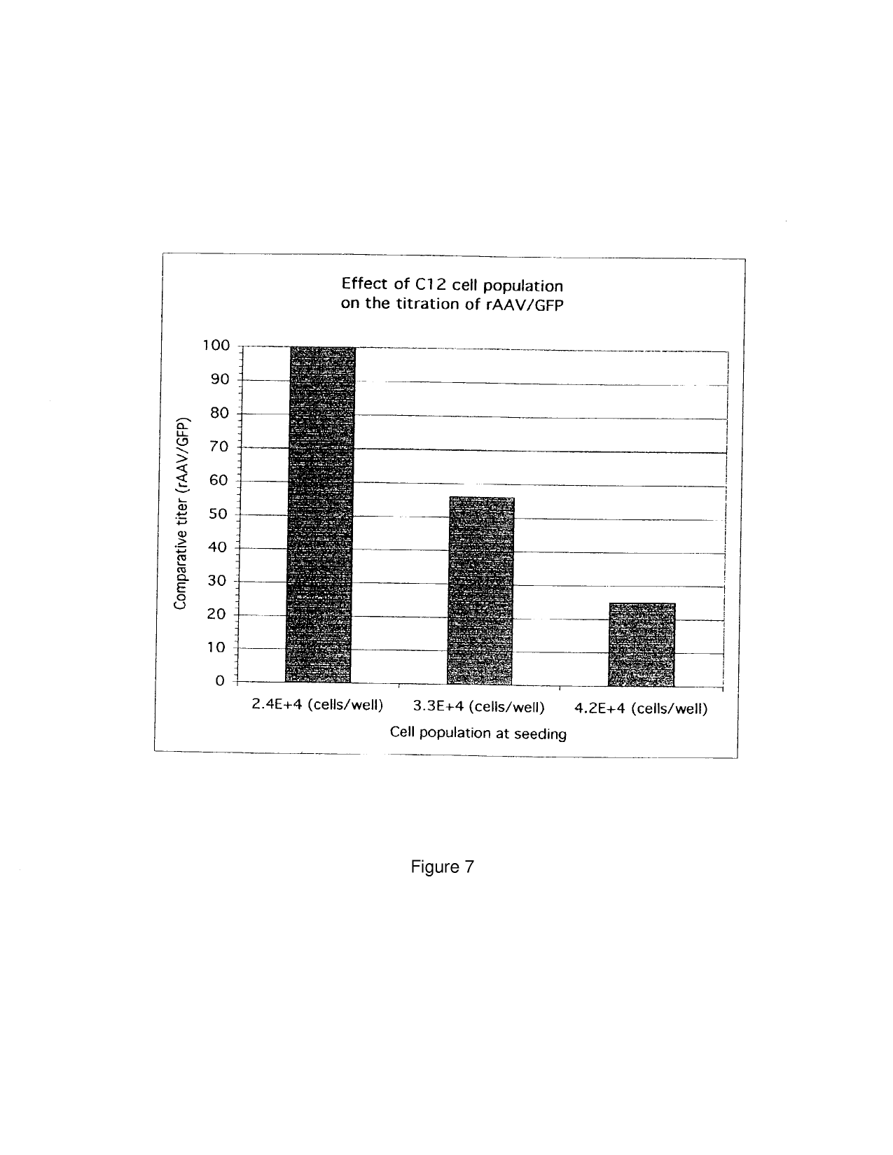

The number of infectious rAAV contained in the cell lysate from the producer cells was determined by infection of a second cell line with the rAAV. The cell line used for this purpose was C12. To determine the effect of seeding density of C12 cells for this assay, C12 cells were plated at various seeding densities and used for analysis of rAAV-GFP production following treatment with lysates from 293 producer cells co-infected with rHSV/rc and rHSV/GFP. The results, shown in FIG. 7, demonstrated that optimal sensitivity of the fluorescence assay was obtained from cells seeded at the lowest density, i.e., at 2.4×10⁴ cells/well. At higher initial plating densities, detection sensitivity was reduced to about 55% and 25%, respectively, for cells seeded at 3.3×10⁴ and 4.2×10⁴ cells/well.

### Example 8

### Simultaneous Co-Infection: Comparison of 293 and Vero Cell Lines

This example describes a comparison of the effectiveness of 293 cells as compared with Vero cells for rAAV production. For these assays, 293 cells and Vero cells were treated identically. Results of two separate experiments demonstrated that 293 cells are quantitatively superior to Vero cells for the production of rAAV using the above co-infection protocol with rHSV/rc and rHSV/GFP. In the first experiment, 293 cells produced 1940 i.p./cell, whereas under the same conditions, Vero cells produced 480 i.p./cell. In the second experiment, the respective production levels were 4000 vs. 720 i.p./cell.

### Example 9

### Simultaneous Co-Infection Using Alternate rHSV Vectors

The capsid proteins of a rAAV product are determined by the serotype of the AAV rep used in the construction of the rHSV/rc. The following example provides a method of producing rAAV with capsids based on various AAV serotypes, using the simultaneous co-infection protocol described above.

Construction of rHSV Viruses. Methods have been described for construction of rHSV vectors expressing the AAV-2 rep genes (Conway et al., 1999). The product of such a viral vector, used in conjunction with a rHSV expression virus, is a rAAV with AAV-2 serotype 2 capsid proteins. Alternate recombinant HSV vectors expressing the AAV-2 rep genes and either the AAV-1, AAV-3 or AAV-4 cap genes may be obtained as follows. AAV-1 through AAV-8 may be acquired from American Type Culture Collection. 293 cells are plated onto 60 mm dishes. Twenty four hours later, the 293 cells are infected with the desired AAV serotype (MOI of 500 particles per cell) and then co-infected with Ad (MOI of 10) to produce double-stranded replicative intermediates of the AAV genomes. Twenty four hours after infection, low molecular weight DNA is isolated by Hirt extraction as described by Conway et al., (1997). This DNA then serves as a template for PCR amplification of the AAV cap genes. PCR primers specific for the particular AAV serotype cap genes are used to amplify the cap gene from the appropriate template. These primers have KpnI sites incorporated at their 5′ end. The PCR reaction conditions are standard conditions for denaturing, annealing, and extension that have previously been employed (Conway et al., 1997).

PCR products are separated by gel electrophoresis and purified. PCR products are then sequenced to verify the fidelity of the PCR reaction. The cap gene PCR products are then digested with KpnI. The vector pHSV-106-rc encodes the BamHI region of the HSV-1 tk locus into which the AAV-2 rep and cap genes have been cloned. The vector pHSV-106-rc is the integration vector used to construct d27.1-rc. pHSV-106-rc is also digested with KpnI to cut out the AAV-2 cap gene 3′ of the p40 promoter. AAV cap genes from the serotype of interest are then cloned in frame into this KpnI site. This results in constructs (pHSV-106-rc1, pHSV-106-rc3, and pHSV-106-rc4) in which the entire VP-3 protein (which comprises 90% of the viral capsid) is from the new AAV serotype. The cloning site used for this purpose is downstream of the p40 promoter, ensuring that regulation of cap transcription by the AAV-2 p40 promoter and Rep proteins is not be altered.

To construct the recombinant viruses (e.g., d27.1-rc1, d27.1-rc3, d27.1-rc4, d27.1-rc5, d27.1-rc6, d27.1-rc7, d27.1-rc8) the constructs pHSV-106-rc1, pHSV-106-rc3, and pHSV-106-rc4 are linearized by restriction digest. Each virus is then separately cotransfected into V27 cells along with d27.1-lacz infected cell DNA. This procedure as well as isolation of recombinant clones by limiting dilution has been described in detail and was used to make the original virus, d27.1-rc. (Conway et al., 1999). Restriction digest of recombinant viral DNA and sequencing of the viral genome is used to verify integration of the vector into the HSV genome. The efficiency of the recombinants at producing rAAV is then determined as described for d27.1-rc.

Co-infection Protocols. The simultaneous co-infection protocols described are amenable to use with any rHSV/rc helper virus. While a rHSV/rc based on the capsid proteins of the AAV-2 serotype was used to demonstrate the invention, it is apparent that rHSV vectors based on other AAV serotypes may be employed. Except for choice of AAV serotype (AAV-1, 2, 3, 4, 5, 6, 7, 8, and other possible serotypes) in the rHSV/rc, all other steps in the procedure for production of rAAV would remain the same.

### Example 10

### Highly Productive rHSV-Based Recombinant AAV Manufacturing Process

This example describes a rAAV production method based on co-infection with rHSV in accordance with the invention that provides the advantages of flexibility, scalability, and high yield of infectious rAAV. The rHSV vectors can be readily propagated to high titer in permissive cell lines, both in tissue culture flasks and in bioreactors.

Materials and Methods

Cell lines and viruses. Mammalian cell lines were maintained in Dulbecco's modified Eagle's medium (DMEM, Hyclone) containing 10% (v/v) fetal bovine serum (FBS, Hyclone) unless otherwise noted. Cell culture and virus propagation were performed at 37° C., 5% CO₂ for the indicated intervals.

rHSV-1 vector construction and production. A rHSV-rep2/cap2 vector (originally denoted d27.1-rc) was constructed as previously described. Briefly, rHSV-rep2/cap2 was constructed by homologous recombination of an AAV2 rep and cap gene cassette into the tk locus of the rHSV-1, ICP27-deleted d27.1 vector in which the AAV-2 rep and cap genes are under control of their native promoters (p5, p19 and p40). The rHSV-AAV2/GFP vector was constructed by homologous recombination of a CMV promoter-driven hGFP-neomycin resistance gene cassette, flanked by the AAV-2 ITRs, into the tk locus of the d27.1 vector as described above.

The rHSV-rep2/cap2 and rHSV-AAV2/GFP vectors were propagated on the ICP27-complementing cell line V27. V27 is an ICP27-expressing Vero cell line derivative which harbors approximately one copy of the ICP27 gene per haploid genome equivalent. Infection steps were done in the absence of serum. Vector stocks were propagated either by seeding T225 flasks with 3×10⁷ V27 cells, or 10-stack cell factories with 1.5×10⁹ V27 cells, followed by infecting 24 h post-seeding with either rHSV-rep2/cap2 or rHSV-AAV2/GFP at a MOI of 0.15. rHSV vectors were harvested at 72 hours post-infection (h.p.i.) by decanting the supernatant and removing cellular debris by centrifugation (10 min, 4° C., 1100 g). The pellet was discarded and the supernatant was stored at −80° C. rHSV-1 vector stocks were used for rAAV-2 production without further manipulation.

rHSV plaque-forming unit (pfu) assay. rHSV-rep2/cap2 and rHSV-AAV2/GFP vector stocks were quantified by a modified plaque assay. V27 cells (1.5×10⁶ cells/well) were seeded into six well plates and infected 24 h post-seeding with 10-fold serial dilutions of rHSV-1 vector stocks. The cells were fixed at 48 hpi with ice-cold methanol and incubated at −20° C. for 15 min. Wells were washed with 1×PBS (Cellgro), and incubated for 30 min at room temperature in 1×PBS containing 1% bovine serum albumin (BSA, Fisher). Viral plaques were hybridized to a polyclonal rabbit-anti-HSV-1 antibody (Dako, 1:500) in 1×PBS containing 1% BSA and visualized by application of a polyclonal, horseradish peroxidase (HRP)-conjugated rabbit-anti-rabbit IgG antibody (Abcam, 1:1000) and staining with diaminobenzidine tetrachloride (DAB, Pierce). Viral plaques were scored as dark brown spots.

Western blot analysis of Rep expression in rHSV-rep2/cap2-infected cells. T75 flasks were seeded with 1×10⁷ 293 cells, infected 24 h post-seeding with rHSV-rep2/cap2 (MOI 0.5), and harvested at 48 hpi with ice cold 1×PBS (10 mL). Cells were collected by centrifugation (5 min, 4° C., 280 g) and crude lysate was generated by resuspending in RIPA buffer comprising 1×PBS (100 μL) containing 1% (v/v) NP-40, 0.25% (w/v) DOC, 0.1% (w/v) sodium dodecyl-sulfate (SDS), and 1 μg/mL each of the protease inhibitors aprotinin, leupeptin, and pepstatin and 1 mM phenyl methyl sulfonyl fluoride (PMSF). Protein in clarified lysate was denatured by incubation at 100° C. for 10 min in the presence of 2.5% (v/v) β-mercaptoethanol (Sigma).

Proteins were electrophoretically separated on pre-cast 10% SDS-polyacrylamide gels (Bio-Rad) and transferred to nitrocellulose. Rep proteins were detected by application of an anti-rep antibody (American Research Product, Inc. Catalog No. 03-61071) at 1:2000 dilution, followed by a goat anti-mouse HRP-conjugated secondary antibody (Pierce, Catalog No. 31430) at 1:3000 dilution, and detected with SuperSignal West Pico Chemiluminescent Substrate and Enhancer (Pierce).

Western blot analysis of HSV-1 proteins in rAAV2 vector stocks. HSV proteins in rAAV2 vector stocks were separated and transferred to nitrocellulose as described above. HSV proteins were hybridized to a polyclonal rabbit-anti-HSV-1 antibody (Dako, 1:2000), and visualized by application of a polyclonal, horseradish peroxidase (HRP)-conjugated rabbit-anti-rabbit IgG antibody (Abcam, 1:10000), and detected as described above.

rHSV co-infection production of rAAV-2, lysate preparation, and column chromatography. 293 cells were seeded into T75 flasks (1×10⁷ cells) or 10-stack cell factories (8.3×10⁸ cells) and simultaneously co-infected 24 h later with rHSV-rep2/cap2 and rHSV-AAV2/GFP at the indicated MOIs. Cells were harvested 50-52 hpi by pipetting (flasks) or manual agitation (cell factory), collected by centrifugation (10 min, 4° C., 1100 g), and resuspended in lysis buffer (20 mM Tris-HCl, pH 8.0, 150 mM NaCl, 0.5% (w/v) DOC). Crude rAAV2/GFP lysate was generated by three freeze-thaw cycles (−80° C. to 37° C.). Lysate was clarified by centrifugation (10 min, 4° C., 2600 g). Clarified lysate was assayed as indicated and/or partially purified by column chromatography.

Clarified rAAV-2 lysates were treated with Benzonase (50 U/mL, 1 h, 37° C.) and partially purified by heparin affinity column chromatography (STREAMLINE™ Heparin, Amersham Biosciences). Columns (1 mL) were poured, washed with 10 column volumes (CV) of lysis buffer, and loaded with a portion of crude lysates (2.3×10¹⁰ ip/mL). Columns were washed with 10 CV of 1×PBS and eluted with 10 CV of 1×PBS made 0.3 M in NaCl, pH 7.2. Vector-containing fractions were pooled and assayed as indicated.

Green fluorescent cell assay for infectious rAAV. Infectious rAAV2/GFP particles were quantified by a modified single-cell fluorescence assay (Zolotukhin et al., 2002). Ninety-six well plates were seeded with C12 cells (Clark et al., 1995) (1.2×10⁴ cells/well) and infected 24 h later with 10-fold serial dilutions of crude rAAV2/GFP stocks containing Ad5 (MOI of 10). Infectious rAAV2/GFP particles were scored as green fluorescing cells at 50-65 hpi using an optical microscope (Zeiss) and UV arc lamp (Zeiss, 400 nm excitation, 509 nm visualization). rAAV2/GFP stocks were assayed in quadruplicate and the titers of infectious particles (ip) were averaged.

rAAV vector genome titer. Clarified rAAV2/GFP lysate was diluted and incubated in the presence of 100 U/mL DNAse I (Roche) and 250 U/mL Benzonase (Merck) at 50° C. for 1 h. AAV capsid proteins were heat denatured and vector genome copy number assayed directly with quantitative PCR (qPCR) by amplifying a hGFP gene sequence. The forward and reverse primers and probe were designed using Vector NTI 9.0 and purchased from Genomechanix. The hGFP-bearing proviral plasmid pTR-UF11 was used to generate standard curves. The primers generated a 90 base fragment for both viral and plasmid DNA.

Replication competent AAV assay. Replication competent AAV (rcAAV) in clarified lysate was quantified with qPCR by amplifying an intact left ITR-rep junction. Vector DNA was liberated as described above. The amplified sequence spanned the D-region of the wtAAV2 ITR (bases 124-145) through the 5′ end of the Rep2 coding region of wtAAV2 (bases 340-359). The resulting PCR product was 235 bases in length. DNA from the wild type (wtAAV2) proviral plasmid pSub201 (Samulski et al. 1989) was used as a positive control, generating a 242 base PCR product. A FAM-6/BHQ-1 dual-labeled oligonucleotide (521 nm emission, 450-550 nm absorption) probe was used for detection and quantification. The forward primer, reverse primer and probe were designed using Vector NTI 9.0 and purchased from Genomechanix. Replication competent-free rAAV as described by Grimm et al. (1998) was obtained from the University of Florida Vector Core.

Results.

rHSV vector propagation, characterization, and production of rAAV-2 in 293 cells. Recombinant AAV-2 production as a function of input of rep-cap and GOI was investigated by simultaneously infecting 293 cells with rHSV-rep2/cap2 and rHSV-AAV2/GFP. Initially, the rHSV-rep2/cap2 MOI (4) was fixed and the rHSV-AAV2/GFP MOI was varied (1, 2, 4, 8, 16) to titrate the optimum ITR-GOI construct input. Maximal rAAV-2/GFP production was observed at a rHSV-AAV2/GFP MOI of 2-4. An MOI of 2 was selected to minimize vector input and purification burden.

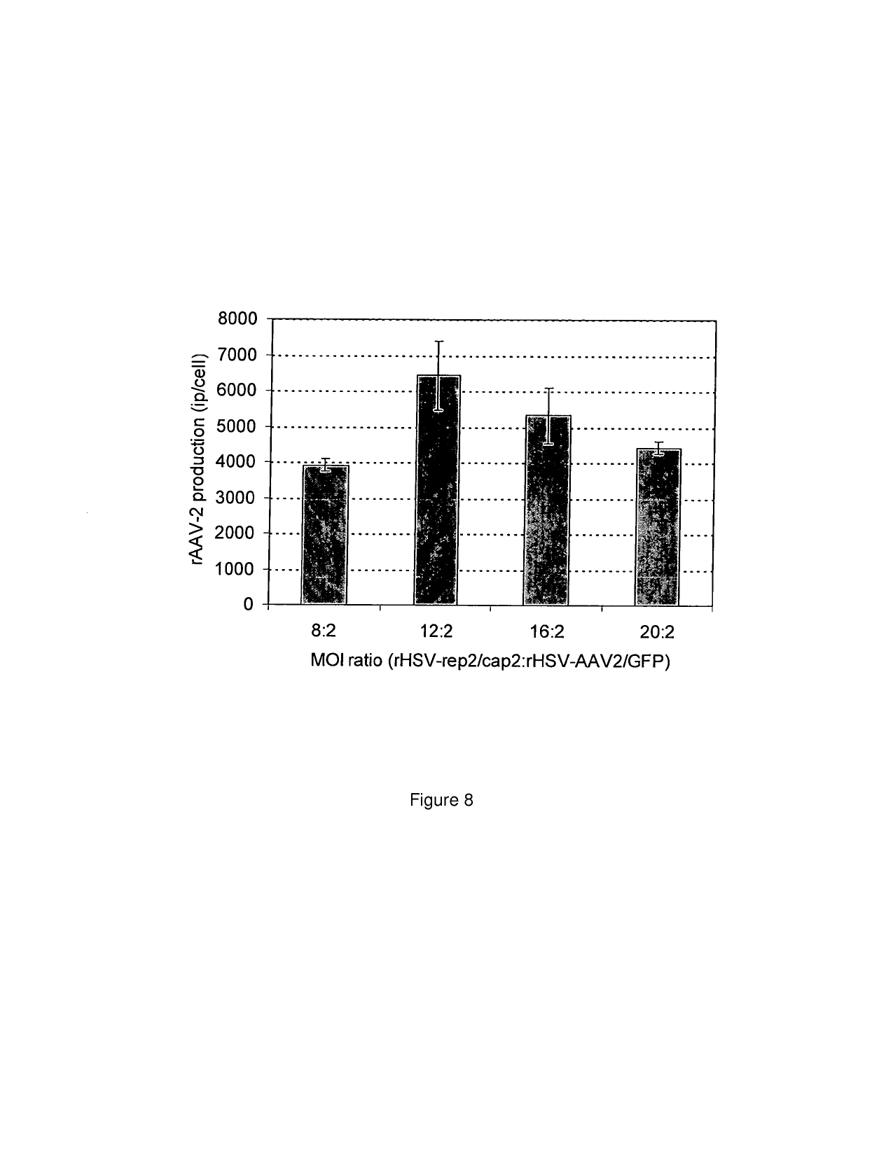

Referring to FIG. 8, the rHSV-rep2/cap2 MOI was then varied (8, 12, 16 20) to determine the optimum rep-cap input. More particularly, FIG. 8 shows rAAV-2 ip/cell production as a function of rHSV-1 vector helper MOI ratio in 293 cells. Harvest time (52 h) and seed density (1×10⁷ cells) were held constant; n=12 for the 12:2 MOI, n=3 for all other MOI ratios. The results indicated a maximum rAAV-2 production of 6400 ip/cell (σ=965), with a viral genome to infectious particle (v.g.:i.p.) ratio of 25, at the optimal rHSV-rep2/cap2 to rHSV-AAV/GFP vector co-infection MOI ratio of 12:2. Studies conducted with an hour or more delay between infection with either rHSV vector resulted in significant declines in per cell yields of rAAV-2.

Optimized yields of rAAV-2 viral genomes in 293 cells were nearly 1×10⁵ per cell, resulting in as many as 7000 rAAV-2 i.p./cell at 1×10⁷ cells. In scaled-up production runs, greater than 4000 i.p./cell yield was achieved at a scale of nearly 10⁹ cells. Accordingly, as shown in Table 1 infra, this new production protocol is capable of attaining per cell v.g. yields on a par with other high-titer rAAV production methods, while yielding a potentially more efficacious vector stock due to lower v.g.:i.p. ratios (Table 1). Use of this method for production of rAAV on a commercial scale could reduce the therapeutic viral genome dose and associated immune response to administration.

TABLE 1
Comparison of rAAV Production Methods and Yields

| rAAV Production | | rAAV | rAAV | vg:ip | |
|---|---|---|---|---|---|
| Method | Cell line | ip/cell | vg/cell | | Reference |
| rHSV-1 co-infection | 293 | 6700^{‡‡} | 97,000^{‡‡} | 15 | This study |
| Triple infection; | 293 | 480 | 126,500 | 260 | Zhang et al. 2001 |
| adenovirus | | | | | |
| Two-plasmid | 293 | 300 | 15,000 | 50 | Grimm et al. 1998 |
| transfection; adenovirus | | | | | Zolotukhin et al. 2002 |
| infection | | | | | |
| Three-plasmid | 293 | 1100 | 100,000 | 90 | Xiao et al. 1998 |
| transfection (Ad helper | | | | | |
| plasmid) | | | | | |
| Three-plasmid | 293 | N/D | 260,000 | N/D | Wustner et al. 2002 |
| transfection (HSV-1 | | | | | |
| helper plasmid) | | | | | |
| Packaging cell line; | 293-GFP- | 1700 | 136,000^{††} | 80 | Qiao et al. 2002 |
| adenovirus infection | 145^{†} | | | | |
| 293 proviral cell line | GFP-92^{†} | 480 | N/D | N/D | Conway et al. 1999 |
| rescue; rHSV-1 infection | | | | | |
| Triple infection; | Sf9 | 33 | 45,000 | 1340 | Urabe et al. 2002 |
| baculovirus | | | | | |
| rHSV-1 amplicon | BHK | 1000 | 100,000 | 100 | Zhang et al. 1999 |
| rHSV-1 co-infection | BHK | 40 | N/D^{‡} | N/D | Booth et al. 2004 |
| rHSV-1 co-infection | BHK | 6454* | 257 | N/D | This study |

^{†}293 proviral cell line
^{††}Calculated using vg:ip of 80 and 5 × 10⁶ 293-GFP-145 cells for best preparation.
^{‡}Capsid titer determined to be 155,000 capsids/cell.
^{‡‡}Average of 6 independent production experiments. See also Example 12.
*See Example 12, infra.

Recombinant HSV vector propagation, characterization, and production of rAAV-2 in V27 cells. In some studies, rHSV-rep2/cap2 and rHSV-AAV2/GFP vectors were propagated on the ICP27-complementing V27 cell line, which is a Vero cell line derivative that harbors a genomic cassette comprising a neomycin resistance gene (neo^{R}) and the ICP27-encoding HSV-1 U_{L}54 gene. V27 cells were infected at an MOI of 0.15 with either the rHSV-rep2/cap2 or rHSV-AAV2/GFP vector. rHSV vectors were recovered by harvesting the cell culture supernatant. Using V27 cells, rHSV vector production was routinely accomplished on a scale of 1.5×10⁹ cells.

Table 2 shows exemplary conditions for optimized production of rAAV-2 in 293 and Vero cells cultured in T75 flasks or in cell factories (CF).

TABLE 2
Optimal rAAV-2/GFP Parameters for Manufacturing rAAV in 293 and Vero Cells.

| cell | seed density | | | | | | |
|---|---|---|---|---|---|---|---|
| line | (cells) | replicates | scale | vg/cell | ip/cell | vg:ip | capsid:vg |
| 293 | 1.0 × 10⁷ | 6 | T75 flask | 96939 +/− 22483 | 6703 +/− 468 | 15 +/− 4.0 | 12 +/− 4.9 |
| 293 | 8.3 × 10⁸ | 4 | CF | 81496 +/− 34860 | 4579 +/− 653 | 17 +/− 4.9 | 4.9 +/− 2.8 |
| Vero | 2.5 × 10⁶ | 4 | T75 flask | N/D | 2118 +/− 211 | N/D | N/D |

MOI ratio was 12:2 and rAAV was harvested at 52 h post-infection for all experiments.

### Example 11

### rAAV-2 Vector Purification

This example describes a purification procedure for rAAV vectors prepared in accordance with the present invention. Results obtained using heparin affinity chromatography and Western blot analysis of rAAV and HSV proteins demonstrate that rAAV-2/GFP stocks generated by the rHSV co-infection method are substantially free of HSV proteins.

Contamination of rAAV stocks with replication-competent AAV (rcAAV) has been recognized as a safety concern and scrutinized since rAAV-mediated transgene delivery was first demonstrated (Hermonat et al. 1984). Several strategies have been pursued to eliminate or reduce rcAAV generation, including p5 promoter removal, intronized AAV genome plasmids, transcription of rep and cap in opposite orientations within the same plasmid, replacement of vector ITRs with a truncated D sequence, and localization of rep and cap on separate plasmids.

The rHSV-rep2/cap2 construct regulates the rep gene from a functional p5 promoter, which might permit rcAAV generation. rAAV-2 vector stocks produced by the HSV co-infection method of the invention were tested for rcAAV contamination using a qPCR method to amplify intact left ITR-rep gene junctions, as described in Materials and Methods. The results showed that rcAAV contamination was not detected by qPCR. rAAV vector stock amplification curves were below the threshold of detection.

Recombinant AAV-2 vector generated by the rHSV co-infection method was next partially purified over a STREAMLINE™ heparin (Amersham Biosciences) affinity column. Crude cellular lysate, containing 0.5% (w/v) deoxycholate (DOC) was generated by three rounds of freezing and thawing. Lysate (2.3×10¹⁰ ip) was applied to the column (1 mL), bound, washed and eluted with PBS made 200 mM, 300 mM, and 500 mM in NaCl.

Crude lysate, column fractions and flow-through were analyzed by Western blot analysis to detect the presence of rAAV structural proteins (VP1, VP2, and VP3) using B1 antibody (Pierce), and to detect HSV proteins using a polyclonal HSV-1 antibody (Dako). Analysis of elution patterns of rAAV-2 and rHSV proteins demonstrated that HSV protein did not bind significantly to heparin in the presence of 0.5% DOC, permitting resolution of HSV protein from rAAV-2 in a single affinity column chromatography step. The Western blot analysis showed rAAV proteins in the crude lysate and fractions but these fractions were devoid of HSV-1 protein as detectable on a Western blot probed with HSV-1 antibody as described. This analysis demonstrates that rAAV vectors propagated and purified in accordance with the inventive methods described herein are substantially free of HSV proteins.

### Example 12

### Suitability of rHSV Co-Infection Method for Production of rAAV in a Variety of Mammalian Cell Lines

In Example 8 above, rAAV production by the rHSV co-infection method was compared in two producer cell lines, i.e., 293 and Vero. Although rAAV yields are lower in Vero than in 293, Vero cells have been approved by the WHO for production of vaccines for human use and therefore may be useful for production of rAAV for human use. This example describes a systematic study of a plurality of cell lines that can be used with the rHSV co-infection procedure to produce rAAV expressing a gene of interest.

This example describes results of studies showing efficient rAAV production in a multiplicity of different mammalian cell lines.

Materials and Methods.

Cell lines were selected for inclusion in the study primarily based on criteria including: infectability by rHSV and Adenovirus; immortalization; acceptable BSL level; and previous use for rAAV production (e.g., 293, BHK, A549, HeLa, etc.). Secondary criteria for selection included ease of culturing, commercial availability, and ability/ease of transfection. Cell lines were selected that met some or all of these criteria, including 293, Vero, BHK-21, A549, HeLa, RD, HT1080, Cos-7, ARPE-19, GeLu, MRC-5, OMK, and WI-38.

The various cell lines were seeded into five replicate T75 flasks. Cells were infected the next day and MOIs based on the cell populations were estimated by harvesting one flask of each cell type. All cells were tested under conditions of receiving a 12:2 MOI ratio of rHSV-AAV rep/cap:rHSV/AAV-GFP.

The results of this analysis demonstrated that rAAV can be produced using the disclosed rHSV co-infection method under the conditions described, in at least ten of the tested cell lines, including 293, Vero, RD, BHK-21, HT-1080, A549, Cos-7, ARPE-19, MRC-5 and WI-38. Robust production of rAAV-GFP (ranging from >1000 to >9000 ip/cell) was achieved in at least three of the previously untested cell lines, as shown in Table 3.

TABLE 3
Production of rAAV by rHSV Co-infection Method in Mammalian Cells

| | Cell density | Average | | |
|---|---|---|---|---|
| Cell line | (at infection) | i.p./cell | Std dev | Replicates |
| 293 | 3.50 × 106 | 9234 | 517 | 4 |
| BHK-21 | 4.50 × 106 | 6454 | 257 | 4 |
| Cos 7 | 9.4 × 105 | 6687 | 617 | 4 |
| Vero | 2.5 × 106 | 2118 | 211 | 4 |
| HT1080 | 3.9 × 106 | 1259 | 62 | 4 |

A particularly advantageous feature of the rHSV co-infection method described herein is its demonstrated flexibility of use with many different cell lines. The method can be applied to any cell line that is permissive for rHSV infection, obviating the many problems associated with cloning and selecting cell lines that are specifically engineered for production of rAAV comprising a particular gene of interest. Different cell lines have different growth characteristics, such as ability to grow in suspension culture, ability to grow in absence of supplementation with animal sera, etc. The disclosed co-infection method allows for the selection of the most advantageous cell types for large-scale production of rAAV vectors.

### REFERENCES

Booth M J, Mistry A, Li X, Thrasher A, Coffin R S. Transfection-free and Scalable Recombinant AAV Vector Production Using HSV/AAV Hybrids. Gene Ther. 2004. 11:829-837.
Buller, R. M. L. 1981. Herpes simplex virus types 1 and 2 completely help adnovirus-associated virus replication. J Virol 40:241-24.
Chiorini, J. A., L. Yang, Y. Liu, B. Safer, and R. M. Kotin. 1997. Cloning of adeno-associated virus type 4 (AAV4) and generation of recombinant AAV4 particles. J Virol 71:6823-6833.
Chiorini, J. A., F. Kim, L. Yang, and R. M. Kotin. 1999. Cloning and characterization of adeno-associated virus type 5. J Virol 73:1309-1319.
Clark, K. R., F. Voulgaropoulou, D. M. Fraley, and P. R. Johnson. 1995. Cell lines for the production of recombinant adeno-associated virus. Hum Gene Ther 6:1329-1341.
Clark, K. R. 2002. Recent advances in recombinant adeno-associated virus vector production. Kidney Internat. 61, Symposium 1:S9-S15.
Conway, J. E., S. Zolotukhin, N. Muzyczka, G. S. Hayward, and B. J. Byrne. 1997. Recombinant adeno-associated virus type 2 replication and packaging is entirely supported by a herpes simplex virus type 1 amplicon expressing Rep and Cap. J Virol 71:8780-8789.
Conway, J. E., C. M. J. ap Rhys, I. Zolotukhin, S. Zolotukhin, N. Muzyczka, G. S. Hayward, and B. J. Byrne. 1999. High-titer recombinant adeno-associated virus production utilizing a recombinant herpes simplex virus type I vector expressing AAV-2 Rep and Cap. Gene Ther. 6:973-985.
Flotte, T. R. and B. J. Carter. 1998. Adeno-associated virus vectors for gene therapy of cystic fibrosis. Methods Enzymol 292:717-732.
Gao, G. P., G. Qu, L. Z. Faust, R. K. Engdahl, W. Xiao, J. V. Hughes, P. W. Zoltick, and J. M. Wilson. 1998. High-titer adeno-associated viral vectors from a Rep/Cap cell line and hybrid shuttle virus. Hum Gene Ther 9:2353-236.
Grimm D, Kern A, Rittner K, Kleinschmidt J A. Novel Tools for Production and Purification of Recombinant Adeno-associated Virus Vectors. Hum. Gene Ther. 1998. 9:2745-2760.
Halbert, C. L., T. A. Standaert, M. L. Aitken, I. E. Alexander, D. W. Russell, and A. D. Miller. 1997. Transduction by adeno-associated virus vectors in the rabbit airway: efficiency, persistence, and readministration. J Virol 71:5932-5941.
Hauswirth, W. W., A. S. Lewin, S. Zolotukhin and N. Muzyczcka. 2000. Production and purification of recombinant adeno-associated virus. Methods Enzymol. 316:743-761.
Hermonat, P. L. and N. Muzyczka. 1984. Use of adeno-associated virus as a mammalian DNA cloning vector: transduction of neomycin resistance into mammalian tissue culture cells. Proc Natl Acad Sci USA 81:6466-6470.
Herzog, R. W., J. N. Hagstrom, S. H. Kung, S. J. Tai, J. M. Wilson, K. J. Fisher, and K. A. High. 1997. Stable gene transfer and expression of human blood coagulation factor IX after intramuscular injection of recombinant adeno-associated virus. Proc Natl Acad Sci USA 94:5804-5809.
Johnston, K. M., D. Jacoby, P. A. Pechan, C. Fraefel, P. Borghesani, D. Schuback, R. J. Dunn, F. I. Smith, and X. O. Breakefield. 1997. HSV/AAV hybrid amplicon vectors extend transgene expression in human glioma cells. Hum Gene Ther 8:359-370.
Iwaki, G. J. Kurtzman, K. J. Fisher, P. Colosi, L. B. Couto, and K. A. High. 1998. Adeno-associated viral vector-mediated gene transfer of human blood coagulation factor IX into mouse liver. Blood 91:4600-4607.
Kaplitt, M. G., P. Leone, R. J. Samulski, X. Xiao, D. W. Pfaff, K. L. O'Malley, and M. J. During. 1994. Long-term gene expression and phenotypic correction using adeno-associated virus vectors in the mammalian brain. Nat Genet. 8:148-154.
Kessler, P. D., G. M. Podsakoff, X. Chen, S. A. McQuiston, P. C. Colosi, L. A. Matelis, G. J. Kurtzman and B. J. Byrne, 1996. Gene delivery to skeletal muscle in sustained expression and systemic delivery of a therapeutic protein. Proc. Natl. Acad. Sci. 93:14082-14087.
Koeberl, D. D., I. E. Alexander, C. L. Halbert, D. W. Russell, and A. D. Miller. 1997. Persistent expression of human clotting factor IX from mouse liver after intravenous injection of adeno-associated virus vectors. Proc Natl Acad Sci USA 94:1426-1431.
Knipe, D. M. 1989. The role of viral and cellular nuclear proteins in herpes simplex virus replication. Adv. Virus Res. 37:85-123:85-123.
Kurtzman, and B. J. Byrne. 1996. Gene delivery to skeletal muscle results in sustained expression and systemic delivery of a therapeutic protein. Proc Natl Acad Sci USA 93:14082-14087.
Matsushita, T., Elliger S., Elliger, C., G. dsakoff, L. llarreal, G. Kurtzman, Y. Iwaki, and P. Colosi. 1998. Adeno-associated virus vectors can be efficienty produced without helper virus. Gene Ther 5:938-945.
McCarthy, A. M., L. McMahan, and P. A. Schaffer. 1989. Herpes simplex virus type 1 ICP27 deletion mutants exhibit altered patterns of transcription and are DNA deficient. J. Virol. 63:18-27.
Mishra, L. and J. A. Rose. 1990. Adeno-associated virus DNA replication is induced by genes that are essential for HSV-1 DNA synthesis. Virology 179:632-639.
Muzczka, N. 1992. Use of adeno-associated virus as a general transduction vector for mammalian cells. Curr Top Microbiol Immunol 158:97-129.
Muzczka, N. and K. I. Berns, 2001. Parvoviridae: The viruses and their replication, pp. 2327-2360. In D. M. Knipe and P. M. Howley (ed.), Fields Virology, Fourth Edition, Lippincott Williams and Wilkins, New York.
Monahan, P. E., R. J. Samulski, J. Tazelaar, X. Xiao, T. C. Nichols, D. A. Bellinger, M. S. Read, and C. E. Walsh. 1998. Direct intramuscular injection with recombinant AAV vectors results in sustained expression in a dog model of hemophilia. Gene Ther 5:40-49.
Qiao C, Wang B, Zhu X, Li J, Xiao X. A novel gene expression control system and its use in stable, high-titer 293 cell-based adeno-associated virus packaging cell lines. J. Virol. 2002. 76:13015-13027.
Rice, S. A. and D. M. Knipe. 1990. Genetic evidence for two distinct transactivation functions of the herpes simplex virus alpha protein ICP27. J. Virol. 64:1704-1715.
Rose, J. A. and F. Koczot. 1972. Adenovirus-associated virus multiplication VII. Helper requirement for viral deoxyribonucleic acid and ribonucleic acid systhesis. J Virol 10:1-8.
Rutledge, E. A., C. L. Halbert, and D. W. Russell. 1998. Infectious clones and vectors derived from adeno-associated virus (AAV) serotypes other than AAV type 2. J Virol 72:309-319.
Samulski, R. J., M. Sally and N. Muzyczka. 1998. Adeno-associated viral vectors. Cold Spring Harbor Laboratory Press, Cold Spring Harbor, N.Y.
Samulski, R. J., A. Srivastava, K. I. Berns, and N. Muzyczka. 1983. Rescue of adeno-associated virus from recombinant plasmids: gene correction within the terminal repeats of AAV. Cell 33:135-143.
Sandri-Goldin, R. M. and G. E. Mendoza. 1992. A herpesvirus regulatory protein appears to act post-transcriptionally by affecting mRNA processing. Genes Dev. 6:848-863.
Snyder, R. O., S. K. Spratt, C. Lagarde, D. Bohl, B. Kaspar, B. Sloan, L. K. Cohen, and O. Danos. 1997. Efficient and stable adeno-associated virus-mediated transduction in the skeletal muscle of adult immunocompetent mice. Hum Gene Ther 8:1891-1900.
Urabe M, Ding C, Kotin R M. Insect Cells as a Factory to Produce Adeno-Associated Virus Type 2 Vectors. Hum. Gene Ther. 2002. 13:1935-1943.
Wagner, J. A., T. Reynolds, M. L. Moran, R. B. Moss, J. J. Wine, T. R. Flotte, and P. Gardner. 1998. Efficient and persistent gene transfer of AAV-CFTR in maxillary sinus. Lancet 351: 1702-1703.
Weindler, F. W. and R. Heilbronn. 1991. A subset of herpes simplex virus replication genes provides helper functions for productive adeno-associated virus replication. J Virol 65:2476-2483.
Weller, S. K. 1991. Genetic analysis of HSV-1 gene required for genome replication. In: Herpes Virus Transcription and Its Regulation. CRC Press, Boca Raton, pp. 105-136.
Wustner, J T, Arnold S, Lock M, Richardson J C, Himes V B, Kurtzman G, Peluso R W. Production of Recombinant Adeno-Associated Type 5 (rAAV5) Vectors Using Recombinant Herpes simplex Viruses Containing rep and cap. Mol. Ther. 2002. 6:510-518.
Xiao, X., J. Li, and R. J. Samulski. 1998. Production of high-titer recombinant adeno-associated virus vectors in the absence of helper adenovirus. J Virol 72:2224-2232.
Xiao, W., N. Chirmule, S. C. Berta, B. McCullough, G. Gao, and J. M. Wilson. 1999. Gene therapy vectors based on adeno-associated virus type 1. J Virol 73:3994-4003.
Ye, X., V. M. Rivera, P. Zoltick, F. J. Cerasoli, M. A. Schnell, G. Gao, J. V. Hughes, M. Gilman, and J. M. Wilson. 1999. Regulated delivery of therapeutic proteins after in vivo somatic cell gene transfer. Science 283:88-91.
Zhang X, de Alwis M, Hart S L, Fitzke F W, Inglis S C, Boursnell M E G, Levinsky R J, Kinnon C, Ali R R, Thrasher A J. High-Titer Recombinant Adeno-Associated Virus Production from Replicating Amplicons and Herpes Vectors Deleted for Glycoprotein H. Hum. Gene Ther. 1999. 10:2527-2537.
Zolotukhin, S., B. J. Byrne, E. Mason, I. Zolotukhin, M. Potter, K. Chesnut, C. Summerford, R. J. Samulski, and N. Muzyczka. 1999. Recombinant adeno-associated virus purification using novel methods improves infectious titer and yield. Gene Ther 6:986-993.

What is claimed is:

1. A method for producing recombinant Adeno-Associated Virus (rAAV), comprising:
(a) simultaneously infecting a mammalian cell, wherein the mammalian cell is selected from the group consisting of a Cos-7 cell and a HT1080 cell, with:
(i) a first replication-defective recombinant herpes simplex virus (rHSV) comprising a nucleic acid sequence operably linked to a promoter wherein the nucleic acid comprises an AAV rep gene and an AAV cap gene; and
(ii) a second replication-defective rHSV comprising a nucleic acid sequence including AAV inverted terminal repeat sequences (ITRs) and a gene of interest, said gene of interest being operably linked to a promoter;
(b) incubating the infected mammalian cell; and
(c) obtaining rAAV from the cell of step (b), wherein the titer of rAAV produced by the cell is between about 1000 and about 9000 infectious particles (i.p.) per cell.

2. The method of claim 1, wherein the mammalian cell is the Cos-7 cell.

3. The method of claim 1, wherein the mammalian cell is the HT 1080-cell.

4. The method of claim 1, wherein the AAV serotype for the cap gene is selected from the group consisting of AAV-1, AAV-2, AAV-3, AAV-4, AAV-5, AAV-6, AAV-7 and AAV-8.

5. The method of claim 4, wherein the AAV serotype for the cap gene is AAV-2.

6. The method of claim 1, wherein the AAV rep and cap genes are integrated into the tk gene of the first rHSV-1.

7. The method of claim 1, wherein the promoter for the rep gene and the cap genes in the first rHSV is a homologous promoter selected from the group consisting of p5, p19, and p40.

8. The method of claim 1, wherein the promoter for the rep gene and the cap gene in the first rHSV is a heterologous promoter selected from the group consisting of a CMV promoter, a SV40 early promoter, a Herpes tk promoter, a metallothionine inducible promoter, a mouse mammary tumor promoter, and a chicken β-actin promoter.

9. The method of claim 1, wherein the second rHSV further comprises a second gene of interest.

10. The method of claim 1, wherein the gene of interest is flanked by the AAV ITRs.

11. The method of claim 1, wherein the gene of interest encodes a protein of therapeutic use in humans.

12. The method of claim 1, wherein the gene of interest encodes a reporter protein that is selected from the group consisting of beta-galactosidase, neomycin phosphoro-transferase, chloramphenicol acetyl transferase, thymidine kinase, luciferase, beta-glucuronidase, xanthine-guanine phosphoribosyl transferase and green fluorescent protein.

13. The method of claim 1, further comprising infecting the cell with at least one additional virus selected from the group consisting of rHSV, rAAV, and recombinant Adenovirus (rAd).

14. The method of claim 1, further comprising transfecting the cell with at least one plasmid DNA.

15. The method of claim 14, wherein the ratio of the first rHSV to the second rHSV when simultaneously infecting the mammalian cell is from about 1:1 to about 10:1.

16. The method of claim 14, wherein the rAAV-producing cell comprising the first rHSV and second rHSV is cultured in a cell factory comprising at least 8×10⁸ cells.

17. The method of claim 1, wherein the rAAV is purified and is substantially free of HSV proteins.

18. A method for producing recombinant rAAV in a mammalian cell, comprising:
(a) simultaneously infecting the mammalian cell, wherein the mammalian cell is selected from the group consisting of a BHK cell and a 293 cell, with:
(i) a first replication-defective rHSV comprising a nucleic acid sequence operably linked to a promoter wherein the nucleic acid comprises an AAV rep gene and an AAV cap gene, wherein the AAV rep and cap genes are integrated into the tk gene of the first rHSV-1; and
(ii) a second replication-defective rHSV comprising a nucleic acid sequence including AAV ITRs and a gene of interest, said gene of interest being operably linked to a promoter;
(b) incubating the infected mammalian cell; and
(c) obtaining rAAV from the cell of step (b), wherein the titer of rAAV produced by the cell is between about 1000 and about 9000 infectious particles (i.p.) per cell.

19. The method of claim 18, wherein the AAV serotype for the cap gene is selected from the group consisting of AAV-1, AAV-2, AAV-3, AAV-4, AAV-5, AAV-6, AAV-7 and AAV-8.

20. The method of claim 18, wherein the AAV serotype for the cap gene is AAV-2.

21. The method of claim 18, wherein the promoter for the rep gene and the cap gene in the first rHSV is a homologous promoter selected from the group consisting of p5, p19, and p40.

22. The method of claim 18, wherein the promoter for the rep gene and the cap genes in the first rHSV is a heterologous promoter selected from the group consisting of a CMV promoter, a SV40 early promoter, a Herpes tk promoter, a metallothionine inducible promoter, a mouse mammary tumor promoter, and a chicken β-actin promoter.

23. The method of claim 18, wherein the second rHSV further comprises a second gene of interest.

24. The method of claim 18, wherein the gene of interest is flanked by the AAV ITRs.

25. The method of claim 18, wherein the gene of interest encodes a protein of therapeutic use in humans.

26. The method of claim 18, wherein the gene of interest encodes a reporter protein that is selected from the group consisting of beta-galactosidase, neomycin phosphoro-transferase, chloramphenicol acetyl transferase, thymidine kinase, luciferase, beta-glucuronidase, xanthine-guanine phosphoribosyl transferase and green fluorescent protein.

27. The method of claim 18, further comprising infecting the cell with at least one additional virus selected from the group consisting of rHSV, rAAV, and recombinant Adenovirus (rAd).

28. The method of claim 18, further comprising transfecting the cell with at least one plasmid DNA.

29. The method of claim 18, wherein the mammalian cell is the BHK cell.

30. The method of claim 18, wherein the mammalian cell is the 293 cell.
